SEQUENCE LISTING (9 sequences)

SEQ ID NO: 1 - DNA, 7213 bp, Artificial Sequence
  pDAWN Addgene 43796 GenBankJN5791211
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgacccatttgctgtccaccagtcatgctagccatatggctgccgcgcggcaccaggccgctgctgtgatgatgatgatgatggctgctgcccatggtatatctccttcttaaagttaaacaaaattatttctagagcaaccattatcaccgccagaggtaaaatagtcaacacgcacggtgttactctagtatataaacgcagaaaggcccacccgaaggtgagccagtgtgactctagtagagagcgttcaccgacaaacaacagataaaacgaaaggcccagtctttcgactgagcctttcgttttatttgatgcctggctctagtagcgatctacactagcactatcagcgttattaagctactaaagcgtagttttcgtcgtttgcagcgccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagctcggcttggagcctgttggtgcggtcatggaattaccttcaacctcaagccagaatgcagaatcactggcttttttggttgtgcttacccatctctccgcatcacctttggtaaaggttctaagctcaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgcaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatctagtatttctcctcttttctagactccgttgtgatgacgcattggtacgcggtatcgggaggttcgaaaatttcgagcgatatcttaaggggggtgccttacgtagaaccccgtaggtcatgcccgaggccggtcctggatggcgcggcggatacgcttgagcaggttttcgtcgagaagcggcttcaaaaccacgtcttttacgccggcctcggcggcccgggtcgagatgttttcgtccggatagccggtgatcaggatcacgggcgtagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggtgcaaaacctttcgcggtatggcatgatagcgcccggaagagagtcaattgagggtggtgaatgtggctagttttcaatcatttgggataccaggacagctggaagtcatcaaaaaagcacttgatcacgtgcgagtcggtgtggtaattacagatcccgcacttgaagataatcctattgtctacgtaaatcaaggctttgttcaaatgaccggctacgagaccgaggaaattttaggaaagaactgtcgcttcttacaggggaaacacacagatcctgcagaagtggacaacatcagaaccgctttacaaaataaagaaccggtcaccgttcagatccaaaactacaaaaaagacggaacgatgttctggaatgaattaaatattgatccaatggaaatagaggataaaacgtattttgtcggtattcagaatgatatcaccgagcaccagcagacccaggcgcgcctccaggaactgcaatccgagctcgtccacgtctccaggctgagcgccatgggcgaaatggcgtccgcgctcgcgcacgagctcaaccagccgctggcggcgatcagcaactacatgaagggctcgcggcggctgcttgccggcagcagtgatccgaacacaccgaaggtcgaaagcgccctggaccgcgccgccgagcaggcgctgcgcgccggccagatcatccggcgcctgcgcgacttcgttgcccgcggcgaatcggagaagcgggtcgagagtctctccaagctgatcgaggaggccggcgcgctcgggcttgccggcgcgcgcgagcagaacgtgcagctccgcttcagtctcgatccgggcgccgatctcgttctcgccgaccgggtgcagatccagcaggtcctggtcaacctgttccgcaacgcgctggaagcgatggctcagtcgcagcgacgcgagctcgtcgtcaccaacacccccgccgccgacgacatgatcgaggtcgaagtgtccgacaccggcagcggtttccaggacgacgtcattccgaacctgtttcagactttcttcaccaccaaggacaccggcatgggcgtgggactgtccatcagccgctcgatcatcgaagctcacggcgggcgcatgtgggccgagagcaacgcatcgggcggggcgaccttccgcttcaccctcccggcagccgacgagatgataggaggtctagcatgacgaccaagggacatatctacgtcatcgacgacgacgcggcgatgcgggattcgctgaatttcctgctggattctgccggcttcggcgtcacgctgtttgacgacgcgcaagcctttctcgacgccctgccgggtctctccttcggctgcgtcgtctccgacgtgcgcatgccgggccttgacggcatcgagctgttgaagcggatgaaggcgcagcaaagcccctttccgatcctcatcatgaccggtcacggcgacgtgccgctcgcggtcgaggcgatgaagttaggggcggtggactttctggaaaagcctttcgaggacgaccgcctcaccgccatgatcgaatcggcgatccgccaggccgagccggccgccaagagcgaggccgtcgcgcaggatatcgccgcccgcgtcgcctcgttgagccccagggagcgccaggtcatggaagggctgatcgccggcctttccaacaagctgatcgcccgcgagtacgacatcagcccgcgcaccatcgaggtgtatcgggccaacgtcatgaccaagatgcaggccaacagcctttcggagctggttcgcctcgcgatgcgcgccggcatgctcaacgattgacaattgatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 2 - DNA, 1407 bp, Artificial Sequence
  pDAWN Opto-mCherry
gtggctagttttcaatcatttgggataccaggacagctggaagtcatcaaaaaagcacttgatcacgtgcgagtcggtgtggtaattacagatcccgcacttgaagataatcctattgtctacgtaaatcaaggctttgttcaaatgaccggctacgagaccgaggaaattttaggaaagaactctcgcttcttacaggggaaacacacagatcctgcagaagtggacaacatcagaaccgctttacaaaataaagaaccggtcaccgttcagatccaaaactacaaaaaagacggaacgatgttctggaatgaattaaatattgatccaatggaaatagaggataaaacgtattttgtcggaattcagaatgatatcaccgagcaccagcagacccaggcgcgcctccaggaactgcaatccgagctcgtccacgtctccaggctgagcgccatgggcgaaatggcgtccgcgctcgcgcacgagctcaaccagccgctggcggcgatcagcaactacatgaagggctcgcggcggctgcttgccggcagcagtgatccgaacacaccgaaggtcgaaagcgccctggaccgcgccgccgagcaggcgctgcgcgccggccagatcatccggcgcctgcgcgacttcgttgcccgcggcgaatcggagaagcgggtcgagagtctctccaagctgatcgaggaggccggcgcgctcgggcttgccggcgcgcgcgagcagaacgtgcagctccgcttcagtctcgatccgggcgccgatctcgttctcgccgaccgggtgcagatccagcaggtcctggtcaacctgttccgcaacgcgctggaagcgatggctcagtcgcagcgacgcgagctcgtcgtcaccaacacccccgccgccgacgacatgatcgaggtcgaagtgtccgacaccggcagcggtttccaggacgacgtcattccgaacctgtttcagactttcttcaccaccaaggacaccggcatgggcgtgggactgtccatcagccgctcgatcatcgaagctcacggcgggcgcatgtgggccgagagcaacgcatcgggcggggcgaccttccgcttcaccctcccggcagccgacgagatgataggaggtctagcatgacgaccaagggacatatctacgtcatcgacgacgacgcggcgatgcgggattcgctgaatttcctgctggattctgccggcttcggcgtcacgctgtttgacgacgcgcaagcctttctcgacgccctgccgggtctctccttcggctgcgtcgtctccgacgtgcgcatgccgggccttgacggcatcgagctgttgaagcggatgaaggcgcagcaaagcccctttccgatcctcatcatgaccggtcacggcgacgtgccgc

SEQ ID NO: 3 - DNA, 40 bp, Artificial Sequence
  YF1_C62S_F
gaaattttaggaaagaactctcgcttcttacaggggaaac

SEQ ID NO: 4 - DNA, 40 bp, Artificial Sequence
  YF1_C62S_R
gtttcccctgtaagaagcgagagttctttcctaaaatttc

SEQ ID NO: 5 - DNA, 19 bp, Artificial Sequence
  T7_terminal_R
gctagttattgctcagcgg

SEQ ID NO: 6 - DNA, 18 bp, Artificial Sequence
  Xpress_R
ccagtcatgctagccata

SEQ ID NO: 7 - DNA, 18 bp, Artificial Sequence
  YF1_seq_1F
gtggctagttttcaatca

SEQ ID NO: 8 - DNA, 17 bp, Artificial Sequence
  cI_middle seq_F
ctctggcgattgaaggg

SEQ ID NO: 9 - DNA, 17 bp, Artificial Sequence
  YF1_middle_seq_F
aacctgttccgcaacgc

(19) United States
(12) Patent Application Publication
(10) Pub. No.: US 2019/0062761 A1
(43) Pub. Date: Feb. 28, 2019

(54) ULTRASOUND METHOD

(71) Applicant: INSTITUTE OF SCIENCE AND TECHNOLOGY AUSTRIA (IST AUSTRIA), Klostemeuburg (AT)

(72) Inventors: Harald JANOVJAK, Wien (AT); Maurizio MORRI, San Carlos, CA (US); Laura Rodriguez HERNANDEZ, Klosterneuburg (AT)

(21) Appl. No.: 16/083,858

(22) PCT Filed: Mar. 10, 2017
(86) PCT No.: PCT/EP2017/055741

§ 371 Date: Sep. 10, 2018

(30) Foreign Application Priority Data
Mar. 11, 2016 (GB) 1604217.8

(51) Int. Cl. C12N 15/63; A61K 41/00; A61K 47/54; A61N 5/06; A61N 7/00

(52) U.S. Cl. CPC C12N 15/635; A61K 41/00; A61K 47/558; C12N 2830/001; A61N 7/00; A61N 2005/0656; A61N 5/062

(57) ABSTRACT

A method of regulating at least one cellular process in one or more cells by exposing the one or more cells to ultrasound, characterised in that the method comprises either (a) introducing into the one or more cells a construct comprising a regulatory transgene capable of regulating the at least one cellular process operably linked to a light-inducible control sequence and (b) inducing the expression of the regulatory transgene using sonoluminescence (SL) or sonochemiluminescence (SCL); or (i) contacting the one or more cells with one or more light-sensitive proteins capable of regulating the at least one cellular process and (ii) regulating the function of the one or more proteins using sonoluminescence or sonochemiluminescence.

### FIELD OF THE INVENTION

The invention relates to regulating at least one cellular process using ultrasound. The invention also relates to treating or preventing diseases using ultrasound. The inventors have termed this “sonobiology”.

### BACKGROUND OF THE INVENTION

Ultrasound (US) stimulation is harnessed to displace matter, catalyze chemical reactions or carry information in a variety of natural and engineered systems. Displacement of matter relies on wave generation in a transducer to move objects or liquids [1, 2], while catalysis takes advantage of pressure and temperature effects to accelerate chemical reactions or permeabilize cells and vesicles [3-6]. In information transmission, sensors receive waves generated by transducers after their interaction with objects in the propagation medium to generate images or measure distances [7, 8]. These and most other well-controlled effects of US stimulation are based on chemical or physical principles, such changes in temperature or pressure. In apparent contrast, biological systems, such as the cells involved in hearing or other forms of mechanosensation, respond to audible and ultrasonic waves with sophisticated multi-faceted programs of bioelectrical and genetic activity [9-12]. Not surprisingly, systems in which a biological process, e.g. gene transcription, protein function or cell signaling, is specifically and selectively controlled by US are currently not known. This gap in technology prevents development of biomedical and sensor systems that would benefit from a combination of the unique properties of US and the unique properties of biological processes. While US allows contactless activation of signals deep in turbid media and with tunable strength by frequency or amplitude adjustment, many biological processes are universal (e.g. activation of mRNA transcription can be in principle coupled to any gene), long-lived (e.g. protein products can be stable for hours to days) and easily detected (e.g. using colorimetric, fluorescent or mass-based methods).

It has been long known that US stimulation of liquids results in the formation cavitating gas bodies, such as microbubbles, that undergo repeated expansion and contraction [13-15](FIG. 1a). Cavitational activity and the associated high-temperature and high-pressure conditions during bubble collapse results in both direct light emission (sonoluminescence (SL)) and decomposition of water molecules with consequent production of radicals, such as hydroxyl (HO.) radicals [16-18]. The radicals generated during bubble collapse can react with chemilumisecent materials, such as luminol, to produce light (sonochemiluminescence, SCL).

In the emerging field of optogenetics, light-activated proteins control key cellular functions non-invasively and with spatial and temporal precision [19, 20].

### SUMMARY OF THE INVENTION

The inventors have surprisingly shown that sonoluminescence (SL) and sonochemiluminescence (SCL) are capable of regulating cellular processes, such expression of target genes, protein functions, cellular morphology and cellular signalling. The inventors have termed this “sonobiology”.

The invention therefore provides a method of regulating at least one cellular process in one or more cells, comprising (a) introducing into the one or more cells a construct comprising a regulatory transgene capable of regulating the at least one cellular process operably linked to a light-inducible control sequence and (b) inducing the expression of the regulatory transgene using sonoluminescence (SL) or sonochemiluminescence (SCL) by exposing the one or more cells to ultrasound. The invention also provides a method of regulating at least one cellular process in one or more cells, comprising (a) contacting the one or more cells with one or more light-sensitive proteins capable of regulating the at least one cellular process and (b) regulating the function of the one or more proteins using sonoluminescence (SL) or sonochemiluminescence (SCL) by exposing the one or more cells to ultrasound.

The invention further provides:
a construct comprising a regulatory transgene capable of regulating at least one cellular process operably linked to a light-inducible control sequence for use in a method of regulating the at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound;
a combination of (a) a construct comprising a regulatory transgene capable of regulating at least one cellular process operably linked to a light-inducible control sequence and (b) and an ultrasound device for use in a method of regulating the at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound using the device;
one or more light-sensitive proteins capable of regulating at least one cellular process or one or more constructs encoding the one or more light-sensitive proteins for use in a method of regulating the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound;
a combination of (a) one or more light-sensitive proteins capable of regulating at least one cellular process or one or more constructs encoding the one or more light-sensitive proteins (b) and an ultrasound device for use in a method of regulating the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound;
a kit for of regulating at least one cellular process in one or more cells comprising (a) a construct comprising a regulatory transgene capable of regulating the at least one cellular process operably linked to a light-inducible control sequence and (b) an ultrasound device;
a kit for regulating at least one cellular process in one or more cells comprising (a) one or more light-sensitive proteins capable of regulating the at least one cellular process or one or more constructs encoding the one or more light-sensitive proteins and (b) an ultrasound device; and
a method of using one or more cells as a sensor for ultrasound intensity, comprising (a) introducing into the one or more cells a construct comprising a regulatory transgene capable of regulating at least one cellular process operably linked to a light-inducible control sequence or contacting the one or more cells with one or more light-sensitive proteins capable of regulating at least one cellular process; (b) exposing the one or more cells to ultrasound to inducing the expression of the regulatory transgene or to regulate the function of the one or more proteins using sonoluminescence (SL) or sonochemiluminescence (SCL); and (c) measuring a change in the at least one cellular process.

### DESCRIPTION OF THE FIGURES

FIG. 1: Sono(chemi)luminescence reaction principle and US device. (a) Reaction mechanism of SL and SCL. Microbubble formation, growth and collapse results in light emission (SL) and production of radicals that can react with a CL reagent (here, luminol or MCLA) for further light emission (SCL). (b and c) Experimental setup (not drawn to scale). 1: black box; 2: PMT; 3: PMT power supply; 4: signal discriminator; 5: amplifier; 6: computer; 7: US transducer; 8: power amplifier; 9: function generator; 10: water supply; 11: flow meter; 12: ice bath; 13: lab sink; 14: thermometer. (d) Representative temperature time course during sonication of water (585 kHz, squares; 1145 kHz, triangles) at 64% power before (solid lines) and after (dashed lines) the implementation of the cooling system. Experiments were stopped when the temperature reached >35° C.

FIG. 2: Measurement chamber for calorimetric method. (a) Temperature of distilled water in the aluminum chamber (200 ml volume capacity, 0.8 mm wall thickness) was measured using a thermometer (0.1° C. accuracy). (b and c) Aluminum chamber connected to the transducer.

FIG. 3: PMT calibration. (a) Emission profiles recorded with the PMT at different LED intensities through ND 3.0 filter. (b) Calibration curve of the PMT with the ND filter from the same data set.

FIG. 4: Reaction scheme for luminol and oxygen radicals. The light emission mechanism is based on the oxidation of luminol, a complex multi-step reaction, which depends on several factors including pH, temperature and ionic strength of the reaction medium and reactive species that can be present in solution and interact with luminol, metal catalyst or hydroxide ions. Dissolving luminol (LH₂) at pH 8 to 14 or in a dipolar aprotic solvents such as dimethylsulfoxide (DMSO, containing O₂) extracts the two acyl-hydrazide protons (pKₐ values, 6.74 and 15.1) from the two cyclic nitrogen atoms, resulting in an intermediate 1 (dianion form, L²⁻), which is readily oxidized by hydrogen peroxide (H₂O₂), hydroxyl radicals (OH) or superoxide radical anion (O₂⁻) to an excited intermediary 2, 3-aminophthalate dianion (3-APA*), accompanied by the loss of molecular nitrogen, N₂. The decay of the excited electronic state to the lower energy level ground state 3 (3-APA) results in photon emission.

FIG. 5: Light generation by SL and SCL. (a) Typical intensities measured for light emission in SL and luminol-catalyzed SCL in water (black bars) and M9 minimal medium (grey bars). (b) Typical raw data traces for data shown in a. (I and II) SL in water or M9 minimal media (pH 7 for both solutions). (III and IV) SCL in water or M9 minimal media after supplementation with luminol (pH 12 for both solutions). Horizontal lines mark continuous US stimulation (585 kHz frequency, 64% amplitude). All solutions were air-saturated and the temperature was maintained at 32±2° C.

FIG. 6: Genetic US detector. (a) Scheme representing the use of SL/SCL to activate gene transcription. E. coli cells harboring the light-activated gene transcription plasmid pDAWN are exposed to US stimulation. Light emission in SCL activates pDAWN, which regulates expression of a marker protein (here, the red fluorescent protein mCherry). (b) Induction of mCherry expression by luminol-catalyzed SCL and pDAWN (red bars). Both US and luminol are required for induction of expression. Expression is moderate compared to light stimulation (also see d). In control experiments, a variant of pDAWN that cannot be activated by light (ΔOPTO, black bars) did not show gene activation by US or light, as expected.

FIG. 7: Effect of US stimulation and reaction solution on E. coli viability. Samples were treated with US, reaction medium or US and reaction medium for 3 h (reaction medium contained 20 mM luminol, pH 9) and then grown overnight at 37° C. Starting conditions are shown in leftmost bars. Black bars: OD600. Grey bars: CFU. Mean values+SD of three independent experiments performed in triplicate are shown.

FIG. 8: Effect of pH on luminol-catalyzed SCL. (a) PMT readout where arrows indicate US stimulation (585 kHz, 64% amplitude, 20 mM luminol). (b) SCL intensities from one representative experiment.

FIG. 9: Reaction scheme for MCLA and oxygen radicals. 2-Methyl-6-(p-methoxyphenyl)-3,7-dihydroimidazol[1,2-a]pyrazin-3-one reacts with superoxide anions (O₂⁻) or singlet oxygen molecules (¹O₂) to lose one hydrogen, via a dioxetanone analogue, which decarboxylates and protonates an excited carbonyl compound to emit light.

FIG. 10: Effect of pH on MCLA-catalyzed SCL. (a) PMT readout where arrows indicate US stimulation (585 kHz, 64% amplitude, 2 μM MCLA). (b) SCL intensities from one representative experiment.

FIG. 11: Induction of mCherry expression by MCLA-catalyzed SCL and pDAWN-V28I. Expression was induced two fold after 2 h of US stimulation (reaction medium contained 2 μM MCLA, pH 7). Mean values+SD of three independent experiments performed in triplicate are shown.

FIG. 12: Effect of M9 salts on MCLA-catalyzed SCL. SCL is reduced by addition of M9 salts (585 kHz, 64% amplitude, pH 7, also compare to Figure S7). One representative experiment is shown.

FIG. 13: Effect of buffers on MCLA-catalyzed SCL. SCL is reduced in three common buffers (HEPES, Tris and potassium phosphate buffer) at physiological pH (585 kHz, 64% amplitude, pH 7, also compare to Figure S7). One representative experiment for each condition is shown.

FIG. 14: Effect of salts on MCLA-catalyzed SCL. SCL is reduced by KH₂PO₄, CaCl₂ and MgCl₂ (585 kHz, 64% amplitude, pH 7, also compare to Figure S7). One representative experiment for each condition is shown.

FIG. 15: Modelled attenuation of light (dashed line) and ultrasound (solid line) as a function of distance from the source. See the Methods of Example 1 for details.

FIG. 16: Time course of pDAWN activation. Emission spectra of mCherry were measured 6, 12 or 18 h after stimulation (dashed, solid, and dotted lines, resp.). Signals reach saturation indicating maturation of mCherry at 12 h after stimulation. Mean raw fluorescence units (RFU) (one representative experiment performed in three wells) are shown.

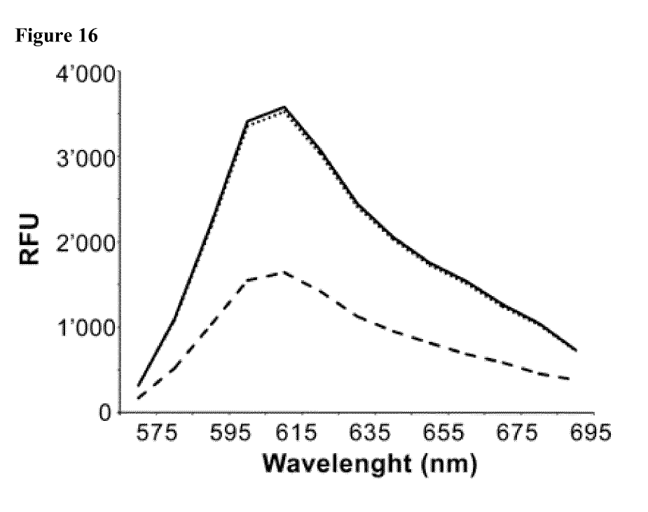

FIG. 17: Morphology changes in M38K cells in response to SCL-Luminol in two chambers configuration. (a) Schematic representation of the reaction mechanism of SCL-luminol in the US reactor to induce EMT-like process in mammalian cells (b) Aspect ratios of M38KOpto-mFGFR1 for each experimental condition (Mean values for >200 individual cells from 6 independent experiments) are shown.

FIG. 18: Morphology changes in M38K cells in response to SCL-Luminol trough a mimic tissue. (a) Schematic representation of the reaction mechanism of SCL-luminol in the US reactor to induce EMT-like process in mammalian cells (b) Aspect ratios of M38K^{Opto-mFGFR1} for each experimental condition (Mean values for >100 individual cells from 3 independent experiments) are shown.

### DESCRIPTION OF THE SEQUENCE LISTING

SEQ ID NOs: 1 to 9 are shown in Tables 5 to 7 in Example 1.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes two or more such polypeptides, or reference to “a polynucleotide” includes two or more such polynucleotides and the like.

All publications, patents and patent applications cited herein, whether supra or infra, are hereby incorporated by reference in their entirety.

### METHODS OF THE INVENTION

### Cellular Processes

The invention concerns regulating at least one cellular process. The method may concern regulating any number of cellular processes, such as 1 or more, 2 or more, 3 or more, 4 or more, 5 or more, 10 or more or even more cellular processes. The invention preferably concerns regulating at least one of (i) expression and/or transcription of a target gene, (ii) function of a protein, (iii) cellular morphology and (iv) cellular signalling. The invention may concern any number and combination of (i) to (iv), such as {i}; {ii}; {iii}; {iv}; {i and ii}; {i and iii}; {i and iv}; {ii and iii}; {ii and iv}; {iii and iv}; {i, ii and iii}; {i, ii and iv}; {i, iii and iv}; {ii, iii and iv}; or {i, ii, iii and iv}.

The method preferably regulates the expression of a target gene and/or the transcription of the target gene. The method may decrease the level of expression and/or transcription of the target gene. The level of expression and/or transcription may be decreased by any amount. For instance, the level of expression and/or transcription may be decreased by at least 10%, at least 30% at least 40%, at least 50%, at least 60%, at least 70%, at least 80%, at least 90% or at least 95%. The method may abolish the expression and/or transcription (i.e. the expression or transcription is decreased by 100%).

The method may increase the level of expression and/or transcription of the target gene. The level of expression and/or transcription may be increased by any amount. For instance, the level of expression or transcription may be increased by at least 10%, at least 30% at least 40%, at least 50%, at least 60%, at least 70%, at least 80%, at least 90%, at least 95% or at least 100%. The level of expression or transcription may be increased by at least a factor of 2, at least a factor of 3, at least a factor of 10, at least a factor of 300, at least a factor of 500, at least a factor of 1000 or more.

The level of transcription can be determined by measuring the amount of mRNA encoded by the target gene. The amount of mRNA can be measured using quantitative reverse transcription polymerase chain reaction (qRT-PCR), such as real time qRT-PCR, northern blotting or microarrays.

The level of expression can be determined by measuring the amount of protein encoded by the target gene. The amount of the protein can be measured using immunohistochemistry, western blotting, mass spectrometry or fluorescence-activated cell sorting (FACS).

The target gene may be involved in any cellular process. The skilled person can select the relevant target gene in order to regulate one or more particular cellular processes. Target genes associated with diseases or disorders are discussed in more detail below.

The method preferably regulates the function of a target protein. The method may decrease the function of the target protein. The function of the target protein may be decreased by any amount. For instance, the function of the target protein may be decreased by at least 10%, at least 30% at least 40%, at least 50%, at least 60%, at least 70%, at least 80%, at least 90% or at least 95%. The method may abolish the function of the target protein (i.e. the function is decreased by 100%).

The method may increase the function of the target protein. The function of the target protein may be increased by any amount. For instance, the function of the target protein may be increased by at least 10%, at least 30% at least 40%, at least 50%, at least 60%, at least 70%, at least 80%, at least 90%, at least 95% or at least 100%. The function of the target protein may be increased by at least a factor of 2, at least a factor of 3, at least a factor of 10, at least a factor of 300, at least a factor of 500, at least a factor of 1000 or more.

Methods for measuring protein function are well known in the art.

Any aspect of cellular morphology may be regulated in accordance with the invention. Cellular morphology may be measured using microscopy, such as fluorescent microscopy, X-ray microscopy or electron microscopy.

The method preferably regulates cellular signalling. Any cellular signalling may be affected. The cellular signalling may be intracrine, autocrine, juxtacrine, paracrine or endocrine. The cellular signalling may take place in the cytoplasm and/or nucleus of the one or more cells. The cellular signalling may involve extracellular receptors, such as G protein-coupled receptors, tyrosine and histidine kinase receptors, integrin receptors, toll gate receptors, ligand-gated ion channel receptors. The cellular signalling may involve intracellular receptors, such as nuclear receptors and cytoplasmic receptors. The cellular signalling may involve second messengers, calcium, lipophilics, nitric oxide and/or redox signalling. The cellular signalling may involve the membrane potential of the one or more cells.

The method may decrease the cellular signalling. The cellular signalling may be decreased by any amount. For instance, the cellular signalling may be decreased by at least 10%, at least 30% at least 40%, at least 50%, at least 60%, at least 70%, at least 80%, at least 90% or at least 95%. The method may abolish the cellular signalling (i.e. the cellular signalling is decreased by 100%).

The method may increase the cellular signalling. The cellular signalling may be increased by any amount. For instance, the cellular signalling may be increased by at least 10%, at least 30% at least 40%, at least 50%, at least 60%, at least 70%, at least 80%, at least 90%, at least 95% or at least 100%. The cellular signalling may be increased by at least a factor of 2, at least a factor of 3, at least a factor of 10, at least a factor of 300, at least a factor of 500, at least a factor of 1000 or more.

Methods for measuring cellular signalling are well known in the art.

### One or More Cells

The method is carried out in more or more cells. The method may be carried out in any number of cells, such 2 or more, 5 or more, 10 or more, 100 or more, 1000 or more, 10⁴ or more, 10⁵ or more, 10⁶ or more, 10⁷ or more, 10⁸ or more, 10⁹ or more, or 10¹⁰ or more cells. The cells may be in vitro. The one or more cells may be present in an in vitro culture. The culture may be present in a culture flask or the wells of a flat plate, such as a standard 96 or 384 well plate. Such plates are commercially available from Fisher scientific, VWR, Nunc, Starstedt or Falcon. The flask or wells may be modified to facilitate culture of the cells, for instance by including a growth matrix. The flask or wells may be modified to allow attachment and immobilization of the one or more cells to the flask or wells. The surface(s) of the flask or wells may be coated with Fc receptors, capture antibodies, avidin:biotin, lectins, polymers or any other capture chemicals that bind to the one or more cells and immobilize or capture them.

Conditions for culturing cells are known in the art. The cells are typically cultured under standard conditions of 37° C., 5% CO₂ in medium supplemented with serum.

The one or more cells in vitro may be any type of cells. Suitable cells for use in the invention include prokaryotic cells and eukaryotic cells. The prokaryotic cell is preferably a bacterial cell. Suitable bacterial cells include, but are not limited to, Escherichia coli, Corynebacterium and Pseudomonas fluorescens. Any E. coli cell with a DE3 lysogen, for example C41 (DE3), BL21 (DE3), JM109 (DE3), B834 (DE3), TUNER, Origami and Origami B, can be used in the invention. Such cells will express a vector comprising the T7 promoter.

Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf9, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other preferred mammalian cells include, but are not limited to, PC12, HEK293, HEK293A, HEK293T, CHO, BHK-21, HeLa, ARPE-19, RAW264.7, M38K and COS cells.

The one or more cells may be in vivo. The one or more cells may be present in any subject. The subject is typically human. However, the subject can be another animal or mammal, such as a research animal, such as a rat, a mouse, a rabbit or a guinea pig, a commercially farmed animal, such as a horse, a cow, a sheep or a pig, or a pet, such as a cat, a dog or a hamster. The one or more cells are preferably present in a tissue. The one or more cells may be respiratory cell(s), cardiovascular cell(s), gastroenterological cell(s), skin cell(s), musculoskeletal cell(s), neurological cell(s), ophthalmological cell(s), genitourinary cell(s), or immune cell(s). The one or more cells may be neoplastic cells or cancer cells. The cancer may be colorectal cancer, prostate cancer, ovarian cancer, lung cancer, central nervous system (CNS) cancer, breast cancer, pancreatic cancer, large intestine cancer or kidney cancer.

The invention therefore provides a construct comprising one or more regulatory transgene capable of regulating at least one cellular process operably linked to a light-inducible control sequence for use in a method of regulating at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound. The invention also provides use of a construct comprising a regulatory transgene capable of regulating at least one cellular process operably linked to a light-inducible control sequence in the manufacture of a medicament for use in regulating at least one cellular process in one or more cells in a subject, wherein the construct is introduced into the one or more cells and the expression of the regulatory transgene is induced using sonoluminescence (SL) or sonochemiluminescence (SCL) by exposing the one or more cells to ultrasound.

The invention also provides a combination of (a) a construct comprising a regulatory transgene capable of regulating at least one cellular process operably linked to a light-inducible control sequence and (b) and an ultrasound device for use in a method of regulating at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound using the device.

The invention also provides one or more light-sensitive proteins capable of regulating at least one cellular process or one or more constructs encoding the one or more light-sensitive proteins for use in a method of regulating the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound. The invention also provides use of one or more light-sensitive proteins capable of regulating at least one cellular process or one or more constructs encoding the one or more light-sensitive proteins in the manufacture of a medicament for regulating the at least one cellular process in one or more cells in a subject, wherein the one or more proteins are contacted with the one or more cells or the one or more constructs are expressed in the one or more cells and the function of the one or more proteins is regulated using sonoluminescence (SL) or sonochemiluminescence (SCL) by exposing the one or more cells to ultrasound.

The invention also provides a combination of (a) one or more light-sensitive proteins capable of regulating at least one cellular process or one or more constructs encoding the one or more light-sensitive proteins (b) and an ultrasound device for use in a method of regulating the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound.

### Transgene Methods

In one embodiment, the invention concerns introducing into the one or more cells a construct comprising a regulatory transgene capable of regulating the at least one cellular process operably linked to a light-inducible control sequence. SL or SCL induces expression of the regulatory transgene. The invention may use a construct comprising two or more regulatory transgenes capable of regulating the at least one cellular process operably linked to a light-inducible control sequence. The invention may use two or more constructs each comprising a regulatory transgene capable of regulating the at least one cellular process operably linked to a light-inducible control sequence.

Suitable expression constructs are known in the art. The terms construct and vector are used herein interchangeably. The construct or vector may be an expression vector. The regulatory transgene is operably linked to a control sequence which is capable of providing for the expression of the regulatory transgene in the one or more cells in response to light.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The term “control sequence” is intended to include promoters, enhancers, internal ribosomal entry sites (IRES), and other expression control elements (e.g. transcription termination signals, such as polyadenylation signals and poly-U sequences). Such control sequences are described, for example, in Goeddel, GENE EXPRESSION TECHNOLOGY: METHODS IN ENZYMOLOGY 185, Academic Press, San Diego, Calif. (1990).

Any light-inducible control sequence may be used in the invention. Suitable control sequences include, but are not limited to those responsive to phytochromes, LOV domains, BLUF domains, cryptochromes, opsins, photoactive yellow proteins, UV-B responsive proteins, vitamin b12 photosensors and fluorescent proteins.

Preferred light-inducible control sequences are
the phytochrome-based enhancer and promoter disclosed in Nat. Biotechnol. 2002 October; 20(10):1041-4.
light-inducible transcription using engineered zinc finger proteins (LITEZ) disclosed in J. Am. Chem. Soc., 2012, 134 (40), 16480-16483;
the light-switchable transactivator LightON disclosed in Nat. Methods (2012) 9: 266-269 (2012);
the synthetic melanopsin-based transcription device disclosed in Science 2011 Jun. 24; 332(6037):1565-8;
EL222-based enhancer and promoterdisclosed in Nat. Chem. Biol. 2014 March; 10(3):196-202;
the cryptochrome-based enhancer and promoter disclosed in Nature. 2013 Aug. 22; 500(7463):472-6;
the ultraviolet B (UVB)-inducible enhancer and promoter disclosed in Nucleic Acids Res. 2013 July; 41(12):e124; and
pDAWN disclosed in Journal of Molecular Biology 2012 Mar. 2; 416(4):534-42.

The light-inducible control sequence is preferably pDAWN, i.e. has the sequence shown in SEQ ID NO: 1, or is a variant thereof. pDAWN has the ability to control the expression of the transgene in a light-inducible manner. The control sequence preferably comprises a polynucleotide sequence which has at least 80%, at least 90%, at least 95%, at least 97%, at least 98% or at least 99% homology based on nucleotide identity with or nucleotide identity with SEQ ID NO: 1 over the entire sequence. There may be at least 80%, for example at least 85%, 90% or 95% nucleotide identity over a stretch of 600 or more, for example 900, 1200, 1500, 1800, 2100, 2400, 3000, 4500 or 6000 or more, contiguous nucleotides (“hard homology”). Homology may be calculated as described below. Polynucleotides are also discussed in more detail below.

The construct or vector is used to deliver the regulatory transgene to the one or more cells. Conventional viral and non-viral based gene transfer methods can be used to introduce the transgene into cells. Non-viral vector delivery systems include DNA plasmids, RNA, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Conventional viral based expression systems could include retroviral, lentivirus, adenoviral, adeno-associated (AAV) and herpes simplex virus (HSV) vectors for gene transfer. Methods for producing and purifying such vectors are know in the art. Since AAV is a DNA virus, the transgene and control sequence used in AAV or rAAV are advantageously DNA.

The construct or vector may be delivered using nanoparticle delivery systems. Such delivery systems include, but are not limited to, lipid-based systems, liposomes, micelles, microvesicles, exosomes, and gene gun. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplated as a means for delivery of a construct or vector in accordance with the invention.

The regulatory transgene is capable of regulating the at least one cellular process as described above. For instance, the transgene may increase expression and/or transcription of the target gene by transactivation. The product of the regulatory transgene may be a transactivator. Alternatively, the transgene may decrease expression and/or transcription of the target gene by transrepression. The product of the regulatory transgene may bind a transcription factor which upregulates the expression and/or transcription of the target gene.

The transgene is preferably the same as the target gene. Expression of the transgene in accordance with the invention increases expression of the target gene in the cell, i.e. increases the amount of the protein encoded by the target gene in the one or more cells.

The transgene may increase the function of the target protein. For instance, the transgene may encode the target protein. In such an embodiment, expression of the transgene increases the amount of the protein in the one or more cells. The transgene may encode the target protein's receptor. In such an embodiment, expression of the transgene increases the amount of the receptor in the one or more cells. Expression of the transgene may decrease the function of the target protein. For instance, the transgene may encode a protein inhibitor of the target protein or an inhibitor of the target protein's receptor.

The transgene may encode a protein which affects the morphology of the one or more cells.

The transgene may increase cellular signalling. For instance, the transgene may encode one or more signalling molecule, such as extracellular or intracellular receptors or G-proteins. In such an embodiment, expression of the transgene increases the amount of the signalling molecules in the one or more cells. Expression of the transgene may decrease cellular signalling.

For instance, the transgene may enocide protein inhibitors of cellular signalling, such as protein inhibitors of extracellular or intracellular receptors.

The skilled person can design suitable systems in accordance with the invention.

The transgene may encode any of the light-sensitive proteins described below.

### Light-Sensitive Protein Methods

In another embodiment, the invention concerns contacting the one or more cells with one or more light-sensitive proteins capable of regulating the at least one cellular process. SL or SCL regulates the function of the one or more light-sensitive protein(s).

Any number of light-sensitive proteins may be used in the invention, such as 2 or more, 3 or more, 5 or more or 10 or more. The method typically uses two or more light-sensitive proteins which interact together. A light-sensitive protein is a protein whose structure and/or function may be altered using light. The light-sensitive protein may be an enzyme. A pair of light-sensitive proteins which interact together in response to light may be used. These pairs may be used as tags to direct other proteins to which they are fused to certain parts of the one or more cells to to bring other proteins to which they are fused together. This allows the light-sensitive proteins to regulate at least one cellular process as described above. Further ways in which light-sensitive proteins can regulate cellular processes are discussed in more detail below.

Light-sensitive proteins can be generated using a light-sensitive domain. A light-sensitive domain may be attached or fused to protein capable of regulating at least one cellular process. The light-sensitive domain is typically covalently linked to the protein. The light-sensitive domain is typically genetically fused to the protein. The light-sensitive domain is genetically fused to the protein if the whole construct is expressed from a single polynucleotide sequence. The coding sequences of the light-sensitive domain and the protein may be combined in any way to form a single polynucleotide sequence encoding the construct. They may be genetically fused in any configuration. They are typically fused via their terminal amino acids. For instance, the amino terminus of the light-sensitive domain may be fused to the carboxy terminus of the protein and vice versa.

The light-sensitive domain may be attached directly to the protein. The light-sensitive domain is preferably attached to the protein using one or more linkers. The one or more linkers may be designed to constrain the mobility of the proteins. Suitable linkers include, but are not limited to, chemical crosslinkers and peptide linkers. Peptide linker are preferred if the light-sensitive domain and protein are genetically fused. Preferred linkers are amino acid sequences (i.e. peptide linkers). The length, flexibility and hydrophilicity of the peptide linker are typically designed such that it does not to disturb the functions of the light-sensitive domain. Preferred flexible peptide linkers are stretches of 2 to 20, such as 4, 6, 8, 10 or 16, serine and/or glycine amino acids. More preferred flexible linkers include (SG)1, (SG)2, (SG)3, (SG)4, (SG)5 and (SG)8 wherein S is serine and G is glycine. Preferred rigid linkers are stretches of 2 to 30, such as 4, 6, 8, 16 or 24, proline amino acids. More preferred rigid linkers include (P)12 wherein P is proline.

Suitable light-sensitive domains for use in the invention include, but are not limited to, phytochromes, LOV domains, BLUF domains, cryptochromes, opsins, photoactive yellow proteins, UV-B responsive proteins, vitamin b12 photosensors, and fluorescent proteins.

Preferred light-sensitive proteins for use in the invention include, but are not limited to, the following.
Channelrhodopsin2, which is a light-driven ion channel from Chlamydomonas rheinhardtii (ChR2) (Proc Natl Acad Sci USA. 2003 Nov. 25; 100(24):13940-5). This may be used to regulate the membrane potential of the one or more cells, especially one or more neurons. It may also be used to regulate ion-based cellular signalling.
Halorhodopsin, which is a light-driven chloride pump from Natronomonas pharaonis (NpHR) (PLoS One. 2007 Mar. 21; 2(3):e299). This may also be used to regulate the membrane potential of the one or more cells, especially one or more neurons. It may also be used to regulate chloride-based cellular signalling.
Opsin-G-protein-coupled receptor (GPCR) chimeras (optoXRs; Nature. 2009 Apr. 23; 458(7241): 1025-9). These may be used to regulate cellular signalling through GPCR pathways.
LiGluR, a light-activated ionotropic glutamate receptor (Nat Chem Biol. 2006 January; 2(1):47-52). This can be used to regulate cellular signalling via glutamate.
light-oxygen-voltage-domain2 (LOV2)-Rac1 (Nature. 2009 Sep. 3; 461(7260):104-8). This allows the regulation of cell morphology and motility.
bPAC, a photoactivated adenylyl cyclase from Beggiatoa (J Biol Chem. 2011 Jan. 14; 286(2):1181-8). This is an adenylyl cyclase directly linked to a BLUF (blue light receptor using FAD) type light sensor domain. It may be used to regulate cellular signalling through cAMP.
BLUF domains (Trends Biochem Sci. 2002 October; 27(10):497-500). BLUF domains are a FAD-binding domain involved in sensory transduction in microorganisms.
euPAC, a photoactivated adenylyl cyclase from Euglena gracilis (Nat Methods. 2007 January; 4(1):39-42). This may be used to regulate cellular signalling through cAMP.
PhyB (phytochrome B) and PIF6 (phytochrome interacting factor 6)/PIF3. (Nature. 2005 Nov. 24; 438(7067):441-2). These two proteins interact in a light sensitive manner. When fused to other proteins, they can be used as light-inducible protein interaction modules. They can be used to regulate protein function and cellular signalling. They can be used to precisely reshape and direct cell morphology.
CRY2 (Cryptochrome 2), and CIB1. These are light-inducible protein interaction modules based on Arabidopsis thaliana cryptochrome 2 and CIB1 that require no exogenous ligands and dimerize on blue light exposure with sub-second time resolution and subcellular spatial resolution (Nat Methods. 2010 December; 7(12): 973-975). They can be used to regulate protein translocation, transcription, and Cre-mediated DNA recombination using light.
Dronpa, a fluorescent protein from Pectiniidae (Science. 2012 Nov. 9; 338(6108):810-4).

This can be fused to proteins and used to regulate their function in one or more cells in a light-sensitive manner.
FKF1 (flavin-binding kelch repeat f-box1), and Gi (Nat Biotechnol. 2009 October; 27(10):941-5). These are protein tags whose interaction is controlled by blue light. They can be used to dimerise proteins to which they are fused in a light sensitive manner. They can regulate protein function and cellular signalling.
TULIPs (Nat Methods. 2012 Mar. 4; 9(4):379-84). These are tunable light-inducible dimerization tags (TULIPs) based on a synthetic interaction between the LOV2 domain of Avena sativa phototropin 1 (AsLOV2) and an engineered PDZ domain (ePDZ). TULIPs can recruit proteins to diverse structures in living yeast and mammalian cells, either globally or with precise spatial control using a steerable laser. They can be used to regulate protein function, cellular signalling and/or cell morphology.
UV-resistance locus 8 (UVR8)—Constitutively photomorphogenic 1 (COP1): UVR8-COP1. This an optogenetic system based on the ultraviolet-B-dependent interaction of the Arabidopsis ultraviolet-B photoreceptor UVR8 with COP1 that can be performed in visible light background. The system can be used to induce nuclear accumulation of cytoplasmic protein fused to UVR8 in cells expressing nuclear COP1 and to recruit a nucleoplasmic protein fused to COP1 to chromatin in cells expressing UVR8-H2B (Nucleic Acids Res. 2013 July; 41(12):e124). It can therefore be used to regulate expression and/or transcription of a target gene.
photoactive yellow protein (PYP) (J Mol Biol. 2010 May 28; 399(1):94-112). This a photocontrolled bZIP-type DNA binding protein that is a hybrid of the prototypical homodimeric bZIP protein GCN4 and photoactive yellow protein (PYP), a blue-light-sensitive protein from Halorhodospira halophila. It can be used to regulate the expressionand/or transcriptin of a target gene.
Opto-mFGFR1, a modified fibroblast growth factor receptor that is exclusively activated by blue light through the light-oxygen-voltage-sensing (LOV) domain of aureochrome 1 from Vaucheria frigida [21, 22].

These proteins may be used to regulate at least one cellular process in the one or more cells.

Sequences of these proteins can be found at the following GenBank and Uniprot accession numbers.

### Channelrhodopsin 2—UNIPROT: B4Y 105

### Halorhodopsin—UNIPROT: P15647

Opsin-G-protein coupled receptor—GenBank: P02699
LiGluR—Protein Data Bank: iGlu R6-methyl glutamate, 1SD3/azobenzene-MAG
Light-oxygen-voltage-domain2 (LOV2)-Rac1—GenBank: CP002686.1
bPAC—GenBank: GU461306.2

### BLUF—UNIPROT: A7BT71; GenBank: GU461307.1

euPAC—GenBank: AB031226.1
PHYB (Arabidopsis thaliana)—UNIPROT: P14713
CRY2 (Arabidopsis thaliana)—GenBank: 839529

### DRONPA—UNIPROT: Q5TLG6

FKF1 flavin-binding, kelch repeat, fbox 1—GenBank: 843133

### TULIPs—GenBank: AAC05083.1

Arabidopsis thaliana LOV1 (LOV) gene—GenBank: EF472599.1
UVB-resistance protein UVR8 (UVR8) mRNA—GenBank: AF130441.1

### PYP—UNIPROT: P16113

### Opto-mFGFR1—AddGene: 58745

The amino acid sequences of the light-sensitive domains and light-sensitive proteins are known in the art and may be found in the citations and GenBank and Uniprot accession numbers above. The method may involve the use of a variant of these domains or proteins. Variants are proteins which differ in their sequence from the original domain or protein, but retain the function of the domain or protein.

Over the entire length of the domain or protein, the variant will preferably be at least 80% homologous to the domain or protein sequence based on amino acid identity over its entire length or have at least 80% amino acid identity to the domain or protein over its entire length. More preferably, the variant may be at least 85%, at least 90% and more preferably at least 95%, 97% or 99% homologous based on amino acid identity to or identical to the domain or protein over the entire sequence. There may be at least 80%, for example at least 85%, 90% or 95%, amino acid identity over a stretch of 200 or more, for example 300, 400, 500, 600, 700, 800, 1000, 1500 or 2000 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

Amino acid substitutions may be made to the domain or protein, for example up to 1, 2, 3, 4, 5, 10, 20, 30, 50, 100 or 200 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 2
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 3
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the domain or protein may additionally be deleted from the polypeptides described above. Up to 1, 2, 3, 4, 5, 10, 20, 30 or 50 residues may be deleted, or more.

Variants may include fragments of the domain or protein. Fragments may be at least 50, at least 100, at least 200, or at least 300 amino acids in length. One or more amino acids may be alternatively or additionally added to the polypeptides described above.

The domain or proteins discussed above may form part of a fusion protein (or chimeric protein) comprising one or more heterologous protein domains, such as about or more than about 1, 2, 3, 4, 5, 6, 7, 8, 9, 10. A fusion protein may comprise any additional protein sequence and optionally a linker sequence between any two domains. Examples of protein domains that may be fused to the proteins discussed above include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A light-sensitive domain or protein may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP 16 protein fusions. Additional domains that may form part of a fusion protein are described in US20110059502.

The one or more light-sensitive domain or proteins may be fused to a fragment crystallizable region (Fc region). The Fc region may be from any of the types of subject discussed below. Fc region is preferably human. The Fc region may derived from any isotype of antibody, such as IgA, IgD, IgG, IgE or IgM.

The light-inducible domains, protein interaction modules and tags disclosed above are typically fused to proteins which will effect the at least one cellular function in accordance with the invention.

The one or more light-sensitive proteins may be labelled with a detectable label. The detectable label may be any suitable label which allows the protein(s) to be detected. Suitable labels include, but are not limited to, fluorescent molecules, radioisotopes, e.g. ¹²⁵I, ³⁵S, enzymes, antibodies, antigens, polynucleotides and ligands such as biotin. The label is preferably a tracer that is suitable for positron emission tomography (PET), such as fluorine (¹⁸F). The label is preferably a tracer suitable for magnetic resonance imaging (MRI), such as fluorine (¹⁹F).

The protein(s) used in the invention may be made in any way. They may be made recombinantly using any of the expression constructs/vectors and in vitro cells discussed above.

Alternatively, the protein(s) may be made by solid-phase peptide synthesis (SPPS) is a preferred technique. This involves formation of the peptide on small solid beads. Using SPPS, the protein remains covalently attached to a bead during synthesis. The protein is synthesised using repeated cycles of coupling-washing-deprotection-washing. In particular, the free N-terminal amine of a solid-phase attached protein is coupled to a single N-protected amino acid unit. This unit is then deprotected, revealing a new N-terminal amine to which a further protected amino acid is attached. These steps are repeated until the protein is complete. The polypeptide is then cleaved from the beads using a suitable reagent.

Suitable protecting groups, reagents, solvents and reaction conditions for SPPS are well known to those skilled in the art and as such conditions can be determined by one skilled in the art by routine optimization procedures.

The invention encompasses using any pharmaceutically acceptable salt of a protein described herein. Said pharmaceutically acceptable salts include, for example, mineral acid salts such as chlorides, hydrochlorides, hydrobromides, phosphates, sulfates, and the like; and the salts of organic acids such as acetates, propionates, malonates, benzoates, and the like; and salts of monocationic metal ions such as sodium and potassium and the like; and salts of bases such as ammonia. A hydrochloride salt or an acetate salt is preferred.

Pharmaceutically acceptable salts of proteins can be prepared by any suitable technique. Typically, salification involves reaction of the protein or a salt thereof with a suitable reagent, typically acid, to obtain the pharmaceutically acceptable salt selected.

For example, a hydrochloride salt of a protein can be prepared by initially cleaving the protein from the solid phase using trifluoroacetic acid. The protein will thus initially be a trifluoroacetate salt. The trifluoroacetate salt can then be converted into a hydrochloride salt by any known technique, such as ion exchange on a suitable column using hydrochloric acid as an eluent.

The protein or protein salt products can be purified, where required, by any suitable technique. High pressure liquid chromatography (HPLC) can be used, for example.

The term “protein” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237. This approach involves making pseudopolypeptides containing changes involving the backbone, and not the orientation of side chains. Meziere et al (1997) show that, at least for MHC class-II and T helper cell responses, these pseudopolypeptides are useful. Retro-inverse proteins, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the proteins may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH₂→—NH(Me) or —N(Me)₂).

Proteins according to the invention may also include variants that increase or decrease the protein's half-life in vivo. Examples of analogues capable of increasing the half-life of proteins used according to the invention include peptoid analogues of the peptides, D-amino acid derivatives of the peptides, and peptide-peptoid hybrids. A further embodiment of the variant proteins used according to the invention comprises D-amino acid forms of the protein. The preparation of proteins using D-amino acids rather than L-amino acids greatly decreases any unwanted breakdown of such an agent by normal metabolic processes, decreasing the amounts of agent which needs to be administered, along with the frequency of its administration.

Modifications as described above may be prepared during synthesis of the peptide or by post-production modification, or when the protein is in recombinant form using the known techniques of site-directed mutagenesis, random mutagenesis, or enzymatic cleavage and/or ligation of nucleic acids.

The proteins described herein may also be modified to improve physicochemical characteristics. Thus, for example, original amino acid sequences may be altered to improve their solubility, and accordingly a protein of the invention having a variant sequence will preferably be more soluble than a protein having the corresponding original amino acid sequence under equivalent conditions. Methods for evaluating the solubility of proteins are well known in the art.

Methods for introducing one or more proteins into one or more cells are known in the art and are discussed in more detail below.

The method preferably comprises expressing the one or more light-sensitive proteins in the one or more cells. The method may comprise introducing into the one or more cells one or more constructs comprising one or more polynucleotides(s) which encode the one or more light-sensitive proteins operably linked to one or more control sequences. Different polynucleotides encoding different light-sensitive proteins may be present in different constructs/vectors or the same construct/vector. If present in the same construct/vector, the different polynucleotides encoding different light-sensitive proteins may be operably linked to different control sequences or the same control sequence.

Suitable expression constructs/vectors are known in the art and described above. The construct/vector may contain any of the light-inducible control sequences discussed above. Alternatively, suitable control sequences include those that direct constitutive expression of nucleotide sequences or those that direct expression of the nucleotide sequence only in certain cell types. Control sequences may direct expression in a temporal-dependent manner, such as in a cell-cycle dependent or developmental stage-dependent manner, which may or may not also be tissue or cell-type specific. In some embodiments, a construct/vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the (3-actin promoter, the phosphoglycerol kinase (PGK) promoter, and the EF1a promoter. Also encompassed by the term “control sequence” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression construct/vector can depend on such factors as the choice of the cell to be transformed, the level of expression desired, etc. With regards to control sequences, mention is made of U.S. patent application Ser. No. 10/491,026. With regards to promoters, mention is made of PCT publication WO 2011/028929 and U.S. application Ser. No. 12/511,940.

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide is typically a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

Polynucleotide sequences may be derived and replicated using standard methods in the art, for example using PCR involving specific primers. It is straightforward to generate polynucleotide sequences using such standard techniques.

The amplified sequences may be incorporated into a recombinant replicable vector such as a cloning vector. The vector may be used to replicate the polynucleotide in a compatible host cell. Thus polynucleotide sequences may be made by introducing the polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

Forms in which the polynucleotide sequence may be delivered to the cell are discussed in more detail above.

### Ultrasound

The invention of the invention comprises using sonoluminescence (SL) or sonochemiluminescence (SCL) by exposing the one or more cells to ultrasound to inducing the expression of the regulatory transgene or regulate the function of the one or more light-sensitive proteins. SL and SCL are known in the art and any method may be used in accordance with the invention.

The one or more cells are preferably exposed to ultrasound at a frequency of from about 25 kHz to about 40 MHz, such as from about 25 kHz to about 5 MHz.

The one or more cells are preferably exposed to ultrasound at a power per unit area of from about 0.01 to about 500 W/cm², such as from about 0.1 to about 10 W/cm².

The one or more cells are preferably exposed to ultrasound under conditions which result in a pressure of from about 10 kPa to about 3 MPa, such as from about 40 kPa to about 400 kPa.

Ultrasound devices are known in the art and any type may be used in the invention.

### SCL

The method of the invention preferably uses SCL. The method preferably comprises contacting the one or more cells with a chemiluminescent agent and inducing the expression of the regulatory transgene or regulating the function of the one or more light-sensitive proteins using sonochemiluminescence (SCL) by exposing the one or more cells to ultrasound.

The method may comprise introducing the chemiluminescent agent into the one or more cells. Methods for introducing such agents into cells is known in the art.

The chemiluminescent agent is preferably a luminol, a luciferin, a Cyridina luciferin methoxy analogue (MCLA), a fluorescent solute, an acridinium salt or an oxalate.

The chemiluminescent agent is more preferably one of the following:

Cypridina luciferin analogs
CLA (Synonyms: CLA-phenyl and 2-Methyl-6-phenyl-3,7-dihydroimidazol[1,2-a]pyrazin-3-one)
MCLA (Synonyms: 6-(4-Methoxyphenyl)-2-methyl-3,7-dihydroimidazo[1,2-a]pyrazin-3-one Hydrochloride and 2-Methyl-6-(4-methoxyphenyl)-3,7-dihydroimidazo[1,2-a]pyrazin-3-one Hydrochloride)
FCLA Free Acid (Synonym: 3,7-Dihydro-6-[4-[2-[N′-(5-fluoresceinyl)thioureido]ethoxy]phenyl]-2-methylimidazo[1,2-a]pyrazin-3-one)
Red-CLA (Synonym: [2-[4-[4-[3,7-Dihydro-2-methyl-3-oxoimidazol[1,2-a]pyrazin-6-yl]phenoxy]butyramido]ethylamino]sulforhodamine 101 Firefly luciferin
D-(−)-Luciferin (Synonym: (S)-2-(6-Hydroxy-2-benzothiazolyl)-2-thiazoline-4-carboxylic Acid
   Fluorescent solutes
Fluorescein (Synonym: 3′,6′-dihydroxyspiro[isobenzofuran-1(3H),9′-[9H]xanthen]-3-one)
Eosin
Pyranine (Synonym: trisodium 8-hydroxypyrene-1,3,6-trisulfonate
Pyrene (Synonym: benzo[def]phenanthrene)

### Luminols

Isoluminol (Synonyms: 6-Amino-2,3-dihydro-1,4-phthalazinedione; 4-Aminophthalhydrazide; 4-Aminophthaloylhydrazine)
Luminol (Synonyms: 5-Amino-2,3-dihydro-1,4-phthalazinedione; 3-Aminophthalhydrazide; 3-Aminophthaloylhydrazine
N(4-Aminobutyl)-N-ethylisoluminol (Synonyms: ABEI; 6-[N-4-Aminobutyl)-N-ethylamino]-2,3-dihydro-1,4-phthalazinedione
4-Isoluminol Isothiocynate (Synonyms: 4-Isothiocyanatophthalhydrazide; 2,3-Dihydro-6-isothiocyanato-1,4-phthalazinedione)
   Acridinium salts
Lucigenin (Synonyms: 10,10′-Dimethyl-9,9′-biacridinium Dinitrate and Bis(N-methylacridinium) Nitrate)
MMT (Synonym: 10,10′-Dimethyl-9,9′-biacridinium Bis(monomethyl Terephthalate))

### Therapy

The at least one cellular process regulated in accordance with invention may be associated with a disease or disorder. A cellular process is associated with the disease or disorder if an alteration in the function of the process is associated with the disease or disorder.

The target gene and/or the target protein regulated in accordance with the invention may be associated with a disease or disorder. A target gene and/or target protein is associated with the disease or disorder if an alteration in the function of the gene and/or protein is associated with the disease or disorder. For instance, the function of the gene and/or protein may altered in a subject having the disease or disorder when compared with a subject of the same species and species and sex and of approximately the same age without the disease or disorder. The function of the gene and/or protein may altered in the one or more cells of a subject having the disease or disorder when compared with the function of the gene and/or protein in the same one or more cells of a subject of the same species and sex and of approximately the same age without the disease or disorder (i.e. normal one or more cells).

The disease or disorder may be associated with an increased function of the gene and/or protein. The function of the gene and/or protein may be increased by any amount. For instance, the function may be decreased by at least 10%, at least 30% at least 40%, at least 50%, at least 60%, at least 70%, at least 80%, at least 90% or at least 95% or at least 100% compared with the level of the function in normal one or more cells. The function may be increased by at least a factor of 2 compared with the function in normal one or more cells, such as at least a factor of 3, at least a factor of 10, at least a factor of 300, at least a factor of 500, at least a factor of 1000 or more.

The disease or disorder may be associated with a decreased function of the gene and/or protein. The function of the gene and/or protein may be decreased by any amount. For instance, the function may be decreased by at least 10%, at least 30% at least 40%, at least 50%, at least 60%, at least 70%, at least 80%, at least 90% or at least 95% compared with the level of the function in normal one or more cells. The disease or disorder may have a complete loss of gene and/or protein function (i.e. the function is decreased 100% compared with normal one or more cells). Gene and/or protein function may be measured in any of the ways discussed below. The gene function may be the expression of the gene. The disease or disorder may be associated with an alteration (increase or decrease) in the expression of the gene (an increase or a decrease in the expression of the gene). This is discussed in more detail below.

The gene may be associated with the disease or disorder because of a polymorphism or mutation in the gene. The gene may comprise a missense mutation. Missense mutations change the amino acid sequence of the encoded protein and thus can reduce the function of the protein or abolish it altogether.

The gene may comprise a nonsense mutation. This leads to decay of mRNA and thus a reduction in protein expression.

The gene may comprise a frameshift mutation. The frameshift mutation may be a deletion frameshift mutation or an insertion frameshift mutation. Both types of mutation can decrease the function of the gene or abolish it altogether. Some frameshift mutations can also introduce a pre-mature stop codon and lead to loss of protein expression.

The gene may comprise a deletion inframe mutation. This mutation may also decrease the function of the encoded protein or abolish it altogether.

The mutations discussed above are preferably homozygous.

Mutations in mRNA may be identified using RNA sequencing including next-generation sequencing. Mutations in the gene may be identified using DNA sequencing including next-generation sequencing. This may also be done using Southern blotting, measuring copy-number variation and investigating promoter methylation.

It will be clear from the above that mutations may affect the level expression of the gene, its stability or its ability to function. The disease or disorder may be associated with an increased amount or a decreased amount of the protein encoded by the gene. The disease or disorder may comprise an increased amount or a decreased amount of the protein encoded by the gene compared with the amount in normal one or more cells. The amount of the protein may be increased or decreased by any amount and in particular the % amounts discussed above in relation to gene function.

The amount of the protein can be measured using immunohistochemistry, western blotting, mass spectrometry or fluorescence-activated cell sorting (FACS).

The disease or disorder may be associated with a protein encoded by the gene with an increased function or a decreased function. The disease or disorder may be associated with a protein encoded by the gene with an increased function or a decreased function compared with the protein in normal one or more cells. The function of the protein may be increased or decreased by any amount and in particular the % amounts discussed above in relation to gene function. Any function of the protein may be altered. The function of the protein can be measured using standard assays depending on its function.

Some diseases, such as neurodegenerative diseases, are typically associated with protein misfolding and/or aggregation. Misfolding means that the correct folding of the protein is disrupted and typically results in the protein having an altered three-dimensional structure. Aggregation refers to the ability of the protein to accumulate and clump together. Neurodegenerative diseases are typically associated with plaques or fibrils of aggregated protein in the brain. The disease or disorder may be associated with a protein encoded by the gene which demonstrates an increased or a decreased misfolding. The or disorder may be associated with a protein encoded by the gene with an increased misfolding or a decreased misfolding compared with the protein in one or more normal cells. The misfolding of the protein may be increased or decreased by any amount and in particular the % amounts discussed above in relation to gene function.

Protein misfolding can be measured using any known technique, such as protein nuclear magnetic resonance spectroscopy, circular dichroism, dual polarisation interferometry, vibrational circular dichroism (VCD) techniques, neutron scattering, ultrafast mixing of solutions, photochemical methods, laser temperature jump spectroscopy, proteolysis and optical tweezers. Protein folding is routinely studied using NMR spectroscopy, for example by monitoring hydrogen-deuterium exchange of backbone amide protons of proteins in their native state which provides both the residue-specific stability and overall stability of proteins. Protein misfolding may also be measured using computational modelling.

The disease or disorder may be associated with a protein encoded by the gene which demonstrates an increased or a decreased aggregation. The disease or disorder may be associated with a protein encoded by the gene with an increased aggregation or a decreased aggregation compared with the protein in normal one or more cells. The aggregation of the protein may be increased or decreased by any amount and in particular the % amounts discussed above in relation to gene function.

Protein aggregation may be measured using commercially-available aggregation assays or microscopy to identify the existence of aggregates.

The disease or disorder is preferably associated with a protein encoded by the gene which demonstrates an increased misfolding and/or an increased aggregation. The or disorder may be associated with a protein encoded by the or disorder with an increased misfolding and/or an increased aggregation compared with the protein in one or more normal cells. The misfolding and/or aggregation of the protein may be increased by any amount and in particular the % amounts discussed above in relation to gene function.

It will be clear from the above that mutations may affect the amount of the gene's mRNA. The disease or disorder may be associated with an increased amount or a decreased amount of the gene's mRNA. The disease or disorder may comprise an increased amount or a decreased amount of the gene's mRNA compared with one or more normal cells. The amount of the mRNA may be increased or decreased by any amount and in particular the % amounts discussed above in relation to gene function.

The amount of mRNA can be measured using quantitative reverse transcription polymerase chain reaction (qRT-PCR), such as real time qRT-PCR, northern blotting or microarrays.

The or disorder may be associated with (a) an increased amount of the protein encoded by the gene, (b) an increased amount of the gene's mRNA, (c) a mutation in the gene or (d) an increased misfolding and/or an increased aggregation of the protein encoded by the gene. The or disorder may be associated with (a) a decreased amount of the protein encoded by the gene, (b) a decreased amount of the gene's mRNA, (c) a mutation in the gene or (d) a decreased misfolding and/or a decreased aggregation of the protein encoded by the gene. The or disorder may be associated with any combination of (a) to (d). In particular, the or disorder may be associated with (a); (b); (c); (d); (a) and (b); (a) and (c); (a) and (d); (b) and (c); (b) and (d); (c) and (d); (a), (b) and (c); (a), (b) and (d); (a), (c) and (d); (b), (c) and (d); or (a), (b), (c) and (d). The mutation in (c) may be any of those discussed above.

The disease or disorder may be diagnosed or prognosed in the subject using one or more of its risk factors and symptoms and its association with a gene be determined through knowledge from other subjects. Alternatively, any of the above alterations may be measured in a biopsy obtained from the subject. Such methods may also be carried out on the blood of the subject.

Target genes and target proteins associated with certain diseases or disorders are known in the art. Any of those target genes and/or target proteins may be regulated in accordance with the invention. If the disease is cancer, the target gene is preferably an oncogene.

The cellular morphology and/or cellular signalling regulated in accordance with the invention may be associated with a disease or disorder. Cellular morphology and/or cellular signalling is associated with the disease or disorder if an alteration in the morphology and/or signalling is associated with the disease or disorder. For instance, the morphology and/or signalling may altered in a subject having the disease or disorder when compared with a subject of the same species and sex and of approximately the same age without the disease or disorder. The morphology and/or signalling may altered in the one or more cells of a subject having the disease or disorder when compared with the morphology and/or signalling in the same one or more cells of a subject of the same species and sex and of approximately the same age without the disease or disorder (i.e. normal one or more cells).

### Disease and Disorders

Neoplasia is a cellular morphology that is associated with a disease or disorder. The neoplasm is preferably cancer, i.e. malignant. The cancer may be any of those discussed above.

The disease or disorder is preferably a respiratory, cardiovascular, gastroenterological, skin, musculoskeletal, neurological, ophthalmological, genitourinary, immune system or inflammatory disease or disorder.

Respiratory diseases or disorders include, but are not limited to asthma, respiratory allergy, pneumonia, bronchitis, rhinitis, sinusitis, tracheitis, pharyngitis, croup and otitis.

Cardiovascular diseases or disorders include, but are not limited to angina pectoris, ischemic myocardial infarction, arrhythmia, post-myocardial infarction pain, myocarditis, heart failure and hypertension.

Gastroenterological diseases or disorders include, but are not limited to stomach ulcers, gastritis, liver cirrhosis, pathological states of the oesophagus, gallstones, pancreatitis, constipation, diarrhea, hemorrhoids and fistulae or inflammation of the rectum.

Skin diseases or disorders include, but are not limited to psoriasis, neurodermatitis, dermatitis and atopic dermatitis.

Muscular-skeletal diseases or disorders include, but are not limited to back pain, lumbago, fractures, pulled muscle, torn ligament or tendon, disc prolapse, ischiatitis, osteoporosis, Perthes disease, osteoarthritis, gout, muscle cramp and diseases affecting the integrity of joints, such as age-related disintegration of joints.

Neurological diseases or disorders include, but are not limited to neurodegenerative diseases or disorders, multiple sclerosis, dementia, neuralgias, and stroke. The neurodegenerative disease or disorder is preferably Parkinson's disease (PD), Parkinson's disease dementia (PDD), dementia with Lewy bodies (DLB), multiple system atrophy (MSA), Alzheimer's disease (AD), Pick's disease, frontotemporal dementia, frontotemporal dementia and parkinsonism linked to chromosome 17 (FTDP-17), cortico-basal degeneration, progressive supranuclear palsy, Huntington's disease or amyotrophic lateral sclerosis (ALS; also known as Lou Gehrig's disease and Charcot disease).

Ophthalmological diseases or disorders include, but are not limited to glaucoma, retinopathy, retinal macula degeneration, age-related macula degeneration (AMD), eye infections, and retinal detachment.

Genito-urinary diseases or disorders include, but are not limited to male genital conditions selected from prostatism, impotence, infertility, testicular disease, female conditions selected from pre/postmenstrual pains, fibroids, endometriosis, infertility, myoma, fibromyoma, inflammatory pelvic conditions, diseases of the ovaries, oviduct(s) or cervix and menopause.

Immune system or inflammatory diseases or disorders include, but are not limited to, rheumatoid arthritis, chronic obstructive pulmonary disease, asthma, angina pectoris, osteoarthritis, inflammatory bowel disease, Crohn's disease, ulcerative colitis, psoriasis, multiple sclerosis, systemic lupus erythematosus, artherosclerosis, pathogenic infection, injury or inflammation of the skin, inflammation of internal organs, colitis, gastroenteritis, pneumonia, wound infections, tuberculosis, influenza, sinusitis, chest infections, bronchitis, allergies such as hay fever, and hemorrhoids.

### Prevention or Treatment

The invention may therefore concern a method of treating or preventing in a subject a disease or disorder associated with at least one cellular process in one or more cells, comprising (a) introducing into the one or more cells a construct comprising a regulatory transgene capable of regulating the at least one cellular process operably linked to a light-inducible control sequence and (b) inducing the expression of the regulatory transgene using sonoluminescence (SL) or sonochemiluminescence (SCL) by exposing the one or more cells to ultrasound.

The invention may also concern a method of treating or preventing in a subject a disease or disorder associated with at least one cellular process in one or more cells, comprising (a) contacting the one or more cells with one or more light-sensitive proteins capable of regulating the at least one cellular process and (b) regulating the function of the one or more proteins using sonoluminescence (SL) or sonochemiluminescence (SCL) by exposing the one or more cells to ultrasound.

The disease or disorder may be any of those discussed above. Any of the embodiments discussed above equally apply to these methods.

The invention therefore provides a construct comprising a regulatory transgene capable of regulating at least one cellular process operably linked to a light-inducible control sequence for use in a method of treating or preventing a disease or disorder associated with the at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound. The invention also provides use of a construct comprising a regulatory transgene capable of regulating at least one cellular process operably linked to a light-inducible control sequence in the manufacture of a medicament for treating or preventing a disease or disorder associated with the at least one cellular process in one or more cells in a subject, wherein the construct is introduced into the one or more cells and the expression of the regulatory transgene is induced using sonoluminescence (SL) or sonochemiluminescence (SCL) by exposing the one or more cells to ultrasound.

The invention also provides a combination of (a) a construct comprising a regulatory transgene capable of regulating at least one cellular process operably linked to a light-inducible control sequence and (b) and an ultrasound device for use in treating or preventing a disease or disorder associated with the at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound using the device.

The invention also provides one or more light-sensitive proteins capable of regulating at least one cellular process or one or more constructs encoding the one or more light-sensitive proteins for use in a method of treating or preventing a disease or disorder associated with the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound. The invention also provides use of one or more light-sensitive proteins capable of regulating at least one cellular process or one or more constructs encoding the one or more light-sensitive proteins in the manufacture of a medicament for treating or preventing a disease or disorder associated with the at least one cellular process in one or more cells in a subject, wherein the one or more proteins are contacted with the one or more cells or the one or more constructs are expressed in the one or more cells and the function of the one or more proteins is regulated using sonoluminescence (SL) or sonochemiluminescence (SCL) by exposing the one or more cells to ultrasound.

The invention also provides a combination of (a) one or more light-sensitive proteins capable of regulating at least one cellular process or one or more constructs encoding the one or more light-sensitive proteins (b) and an ultrasound device for use in a method of treating or preventing a disease or disorder associated with the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound.

The method may concern preventing a disease or disorder. In such instances, the method is carried out in a subject without the disease or disorder or in whom the disease or disease is forming. The method is carried out in order to prevent the onset of one or more symptoms of the disease or disorder. This is prophylaxis. In this embodiment, the subject can be asymptomatic. The subject is typically one that is likely to develop the disease or who is genetically predisposed to the disease.

For prevention, a prophylactically effective amount of the construct/vector or one or more proteins is typically administered to the subject. A prophylactically effective amount is an amount which prevents the onset of one or more symptoms of the disease.

The method may concern treating a disease or disorder. In such instances, the method is carried out in a subject with the disease or disorder. The method is carried out in order to reduce or abolish one or more symptoms of the disease or disorder. In this embodiment, the subject is typically symptomatic. The subject is typically one that has been diagnosed as having the disease or disorder or is suspected of having the disease or disorder.

For treatment, a therapeutically effective amount of the construct/vector or one or more proteins is typically administered to the subject. A therapeutically effective amount of is an amount effective to ameliorate one or more symptoms of the disease or disorder. A therapeutically effective amount is preferably an amount effective to abolish one or more of, or preferably all of, the symptoms of the disease or disorder. Doses are discussed in more detail below.

### Pharmaceutical Compositions for Use in the Invention

The construct/vector or one or more proteins may be administered to the subject in a pharmaceutical composition comprising a pharmaceutically acceptable carrier or diluent. The carrier or diluent may be any of those discussed above with reference to the vectors of the invention.

The carrier(s) or diluent(s) present in the pharmaceutical composition must be “acceptable” in the sense of being compatible with the other ingredients of the composition and not deleterious to the recipient thereof. Typically, carriers for injection, and the final formulation, are sterile and pyrogen free. Preferably, the carrier or diluent is water. A pharmaceutically acceptable carrier or diluent may comprise as one of its components thioglycerol or thioanisole.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

The active agents (i.e. the construct/vector or one or more proteins) may be administered are typically present at 0.1% to 50% by weight in the pharmaceutical composition, more preferably at 0.1% to 5% by weight. They may be present at less than 0.1% by weight in the pharmaceutical composition.

The pharmaceutically acceptable carrier or diluent is typically present at 50% to 99.9% by weight in the pharmaceutical composition, more preferably at 95% to 99.9% by weight. The pharmaceutically acceptable carrier or diluents may be present at more than 99.9% by weight in the pharmaceutical composition.

Pharmaceutical compositions include, but are not limited to pharmaceutically acceptable solutions, lyophilisates, suspensions, emulsions in oily or aqueous vehicles, pastes, and implantable sustained-release or biodegradable compositions. Such pharmaceutical compositions may further comprise one or more additional ingredients including, but not limited to, suspending, stabilizing, or dispersing agents. A lyophilisate may comprise one or more of trehalose, thioglycerol and thioanisole. In one embodiment of a pharmaceutical composition for parenteral administration, the active ingredient is provided in dry form (e.g., a lyophilisate, powder or granules) for reconstitution with a suitable vehicle (e.g., sterile pyrogen-free water) prior to parenteral administration of the reconstituted pharmaceutical composition.

The pharmaceutical composition may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable compositions may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parenterally-administrable pharmaceutical compositions which are useful include those which comprise the active ingredient in microcrystalline form, in a liposomal preparation, or as a component of a biodegradable polymer systems. Pharmaceutical compositions for sustained release or implantation may comprise pharmaceutically acceptable polymeric or hydrophobic materials such as an emulsion, an ion exchange resin, a sparingly soluble polymer, or a sparingly soluble salt.

For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical compositions. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral compositions include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release compositions or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Polynucleotides may be present in combination with cationic lipids, polymers or targeting systems.

Uptake of polynucleotide or oligonucleotide constructs may be enhanced by several known transfection techniques, for example those including the use of transfection agents.

Examples of these agents include cationic agents, for example, calcium phosphate and DEAE-Dextran and lipofectants, for example, lipofectamine and transfectam. The dosage of the polynucleotide or oligonucleotide to be administered can be altered.

Alternatively, the active agent may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules. The composition will depend upon factors such as the nature of the active agent and the method of delivery. The pharmaceutical composition may be administered in a variety of dosage forms. It may be administered orally (e.g. as tablets, troches, lozenges, aqueous or oily suspensions, dispersible powders or granules), topically, parenterally, subcutaneously, by inhalation, intravenously, intramuscularly, intralymphatically (such as to lymph nodes in the groin), intrasternally, transdermally, intradermally, epidermally, sublingually, intranasally, buccally or by infusion techniques. The administration may be intratonsillar. The administration may be as suppositories. The administration may be made by iontophoresis. Preferably, the administration is intradermal, epidermal or transdermal. The administration may be made by a patch, such as a microtine patch. Administration is discussed in more detail below.

A physician will be able to determine the required route and means of administration for each particular individual.

The pharmaceutical compositions for use in the invention are preferably provided sealed in a container. The pharmaceutical compositions are typically provided in unit dose form, for example single dose form. They may alternatively be provided in multi-dose form. Where the pharmaceutical composition is a pharmaceutically acceptable solution, the solution may be provided in an ampoule, sealed vial, syringe, cartridge, flexible bag or glass bottle. Where the pharmaceutical composition is a lyophilisate, it is preferably provided in a sealed vial.

The pharmaceutical compositions of the invention will comprise a suitable concentration of each agent to be effective without causing adverse reaction. Where the pharmaceutical composition is for example a lyophilisate, the relevant concentration will be that of each polypeptide following reconstitution. Typically, the concentration of each agent in the pharmaceutical composition when in solution will be in the range of 0.03 to 200 nmol/ml. The concentration of each agent may be more preferably in the range of 0.3 to 200 nmol/ml, 3 to 180 nmol/ml, 5 to 160 nmol/ml, 10 to 150 nmol/ml, 50 to 200 nmol/ml or 30 to 120 nmol/ml, for example about 100 nmol/ml. The pharmaceutical composition should have a purity of greater than 95% or 98% or a purity of at least 99%.

In an embodiment where the invention involves combines therapy, the other therapeutic agents or adjuvants may be administered separately, simultaneously or sequentially. They may be administered in the same or different pharmaceutical compositions. A pharmaceutical composition may therefore be prepared which comprises an agent of the invention and also one or more other therapeutic agents or adjuvants. A pharmaceutical composition of the invention may alternatively be used simultaneously, sequentially or separately with one or more other therapeutic compositions as part of a combined treatment.

### Administration

The construct/vector or one or more proteins may be administered to the subject in any appropriate way. In the invention, the construct/vector or one or more proteins may be administered in a variety of dosage forms. Thus, they can be administered orally, for example as tablets, troches, lozenges, aqueous or oily suspensions, dispersible powders or granules. They may also be administered by enteral or parenteral routes such as via buccal, anal, pulmonary, intravenous, intra-arterial, intramuscular, intraperitoneal, intraarticular, topical or other appropriate administration routes. A physician will be able to determine the required route of administration for each particular subject.

The construct/vector or one or more proteins may be in any of the forms discussed above with reference to the pharmaceutical composition of the invention.

Methods for gene delivery are known in the art. See, e.g., U.S. Pat. Nos. 5,399,346, 5,580,859 and 5,589,466. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells that have been removed from a subject. For example, a polynucleotide, expression cassette or vector of the invention may be introduced into APCs of an individual ex vivo. Cells containing the nucleic acid molecule of interest are re-introduced into the subject such that an immune response can be mounted against the peptide encoded by the nucleic acid molecule. The nucleic acid molecules used in such immunization are generally referred to herein as “nucleic acid vaccines.”

The dose may be determined according to various parameters, especially according to the substance used; the age, weight and condition of the subject to be treated; the route of administration; and the required regimen. Again, a physician will be able to determine the required route of administration and dosage for any particular subject. A typical daily dose is from about 0.1 to 50 mg per kg of body weight, according to the activity of the specific inhibitor, the age, weight and conditions of the subject to be treated and the frequency and route of administration. The dose may be provided as a single dose or may be provided as multiple doses, for example taken at regular intervals, for example 2, 3 or 4 doses administered hourly. Preferably, dosage levels of inhibitors are from 5 mg to 2 g.

Typically polynucleotide or oligonucleotide inhibitors are administered in the range of 1 pg to 1 mg, preferably to 1 pg to 10 μg nucleic acid for particle mediated delivery and 10 μg to 1 mg for other routes.

### Combination Therapy

The construct/vector or one or more proteins is preferably administered in combination with another therapy

The construct/vector or one or more proteins may be used in combination with one or more other therapies intended to treat the same subject. By a combination is meant that the therapies may be administered simultaneously, in a combined or separate form, to the subject.

The therapies may be administered separately or sequentially to a subject as part of the same therapeutic regimen. For example, construct/vector or one or more proteins be used in combination with another therapy intended to treat the one or more disease. The other therapy may be a general therapy aimed at treating or improving the condition of the subject. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with the inhibitor. The other therapy may be a specific treatment directed at the one or more diseases. Such treatments are known in the art.

### Kits of the Invention

The invention also provides kits for of regulating at least one cellular process in one or more cells. In one embodiment, the kits comprises (a) a construct comprising a regulatory transgene capable of regulating the at least one cellular process operably linked to a light-inducible control sequence and (b) an ultrasound device. In another embodiment, the kit comprises (a) one or more light-sensitive proteins capable of regulating the at least one cellular process or one or more constructs encoding the one or more light-sensitive proteins and (b) an ultrasound device. Any of the embodiments discussed above with reference to the methods of the invention equally apply to the kits of the invention.

The kit may additionally comprise one or more other reagents or instruments which enables the method mentioned above to be carried out. Such reagents include means for taking a sample from the subject, suitable buffers, means to express polynucleotides or a support comprising wells on which quantitative reactions can be done. The kit may, optionally, comprise instructions to enable the kit to be used in the method of invention or details regarding subjects on which the method may be carried out. The kit may comprise primers and reagents for PCR, qPCR (quantitative PCR), RT-PCR (reverse-transcription PCR), qRT-PCR (quantitative reverse-transcription PCR) reaction or RNA sequencing.

### Sensor Methods

The invention also provides a method of using one or more cells as a sensor for ultrasound intensity. The method comprises introducing into the one or more cells a construct comprising a regulatory transgene capable of regulating at least one cellular process operably linked to a light-inducible control sequence or contacting the one or more cells with one or more light-sensitive proteins capable of regulating at least one cellular process. Any of the embodiments discussed above may be used.

The one or more cells are then exposed to ultrasound to induce the expression of the regulatory transgene or to regulate the function of the one or more proteins using sonoluminescence (SL) or sonochemiluminescence (SCL). Any of the embodiments discussed above may be used.

A change in the at least one cellular process is then measured. The change can be any of those discussed above and the change can be measured in any of the ways discussed above. The change provides information concerning ultrasound intensity, i.e. the ultrasound is intense enough to generate SL or SCL. The change in the at least one cellular process will only occur if SL or SCL occurs in the one or more cells. As discussed in the Example, many factors affect the threshold for cavitation formation (initial nucleus size and character, pulse features, host fluid composition like content of dissolved gas, density and temperature). This invention allows one or more cells to be used as acoustic (cavitational) sensors on the nano- or micrometer scales.

### EXAMPLES

### Example 1

### Summary

Sound waves serve as information carriers in animal communication, medical diagnostics and object detection. In many natural and engineered systems, sound is converted into short-lived electrical signals by mechanical sensors, such as specialized nerve cells or magnets and coils. Methods to register sound, preferably ultrasound (US), into longer lasting and biologically universal signals, preferably on nano- or micrometer scales, are currently not available but would allow for fundamentally new applications in acoustics and biotechnology. Here, we apply US to specifically activate gene transcription in living cells. We first quantified light emission in sonoluminescence (SL) as well as in luminol- and Cypridina luciferin methoxy analogue (MCLA)-catalyzed sonochemiluminescence (SCL) in a reactor suitable for live-cell experiments.

In balanced reaction conditions for cavitation and cell survival, we obtained light intensities ranging from ˜0.25 to 12 nW/cm². When combined with an optogenetic regulator, US induced protein production in an E. coli expression system. The conversion of sound to gene transcription and protein production with high specificity, yield and persistence will open new avenues in the design of sound sensors and biotechnological devices.

### Introduction

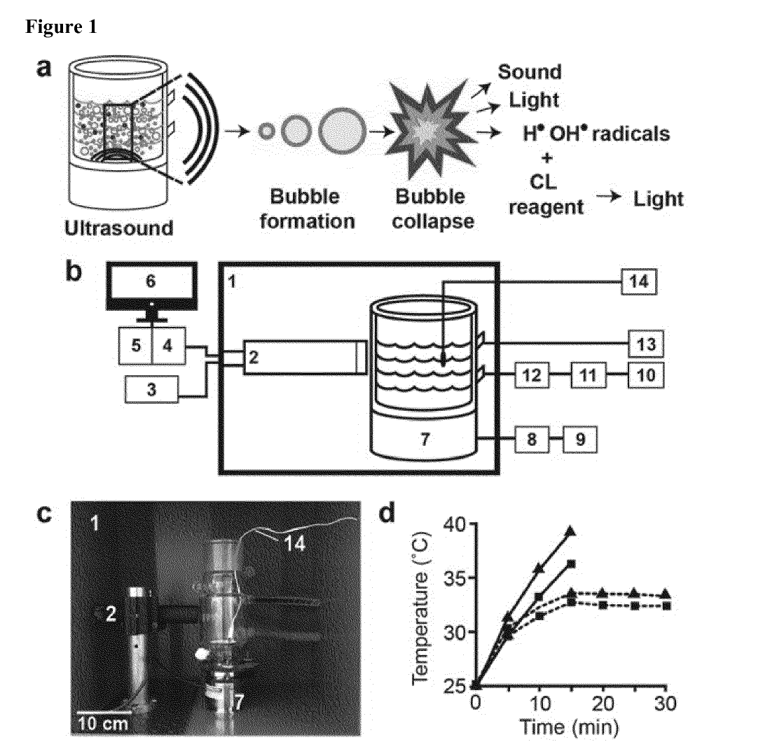

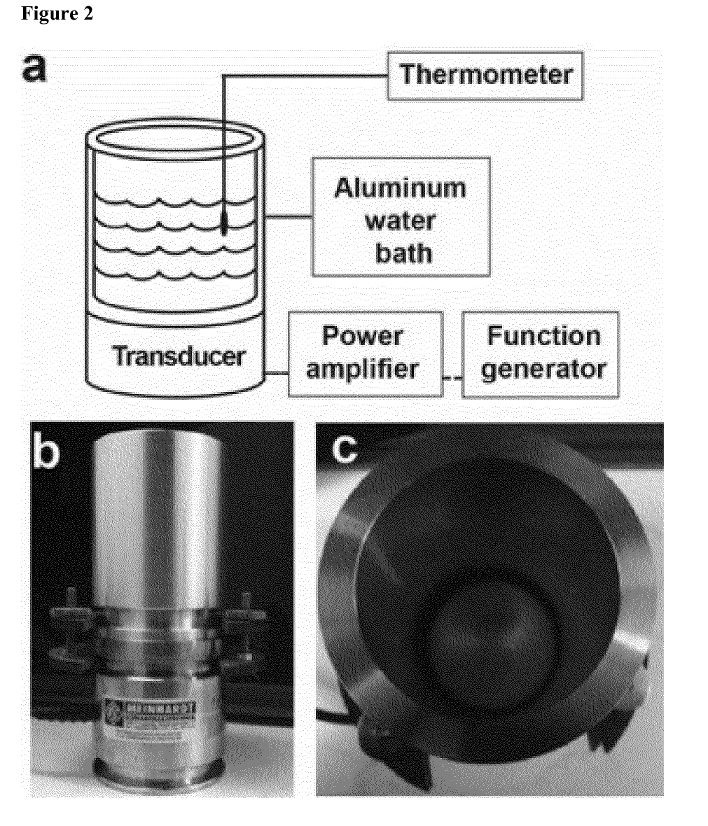

In our experiments, we employed a multi-frequency US device originally developed for sonochemistry (FIG. 1b, c). The device consisted of a piezoelectric transducer with titanium membrane and a glass reaction chamber. Cavitation has been previously observed in a wide range of US frequencies (ν˜25 kHz to 5 MHz). Here, we choose two frequencies (ν=0.585 and 1.145 MHz) in the “extended” US range because these frequencies lie above those employed for lysis of cells (ν˜20 to 200 kHz) and are close to those used in diagnostic and treatment US (ν>1 MHz). We estimated the ultrasonic power of the transducer using a calorimetric method and a water filled aluminum reaction chamber [23] (FIG. 2). We found the power per unit area to range between 0.025 and 0.25 W/cm² for those power settings that are compatible with continuous device operation (up to 64% maximal power; Table 3). These values correspond to pressures of 0.019 to 0.062 MPa (Table 3) and thus both power and pressure in this device are comparable to the upper limits defined for medical diagnostic US devices [15]. When operating the device with the glass reaction chamber at 64% maximal power (see experiments below), we observed a continuous rise in solution temperature that would prevent experiments under conditions that are constant or suited for living cells (FIG. 1d). We thus equipped the device with a cooling system that allowed maintaining constant solution temperature, e.g. at 33±2° C. for experiments with bacteria and mammalian cells (FIG. 1d). Collectively, we established a US device that is calibrated and suited for live cell experiments.

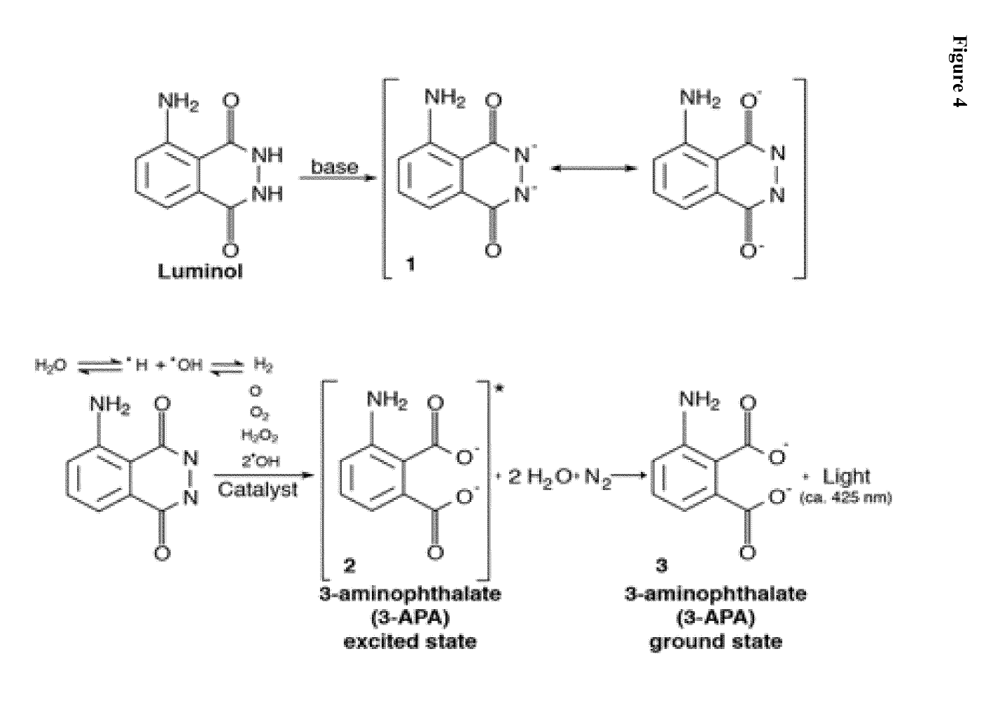

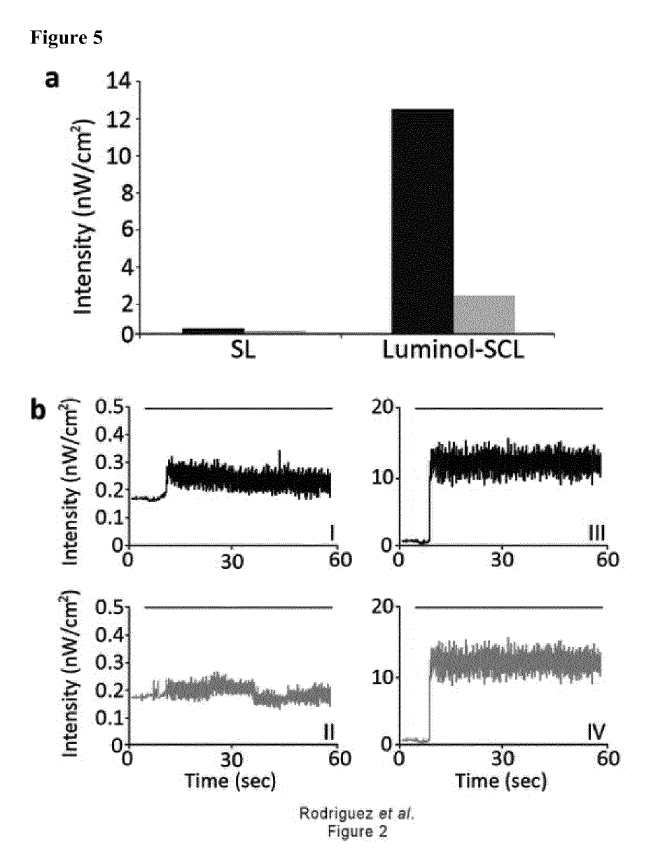

We next measured light emission in SL and SCL in this device. We installed a photomultiplier tube (PMT) on the side of the chamber that we calibrated using a light emitting diode (FIG. 1). For SL, we found light intensity of ˜0.1 nW/cm² in pure water, while the intensity in a chemically-defined bacterial growth medium (M9 minimal medium) was close to the detection limit of the PMT (˜0.02 nW/cm²). These light intensities are at least three orders of magnitude below the intensities known to activate biological processes through genetically-encoded [21, 24, 25] or semi-chemical [26] optogenetic approaches. Thus we concluded that under these experimental conditions a combination of SL and optogenetics is unlikely to be fruitful. In previous studies, increased light emission was observed upon addition of chemiluminescence agents, such as luminol [27, 28], which scavenge radical products during cavitation for photon generation (FIG. 4). After luminol addition and adjustment of pH, we observed intensities of 2 to 12.5 nW/cm² in water and M9 medium (FIG. 5). The light emitted was bright enough to be seen with the naked, dark-adapted eye and approached intensities useful for experiments with optogenetics. Larger emission in SCL compared to SL at the same power can be explained by the observation that SCL depends on radicals produced in low temperature bubbles, whereas SL is generated from high temperature bubbles. This results in a large active-bubble population inducing SCL [29].

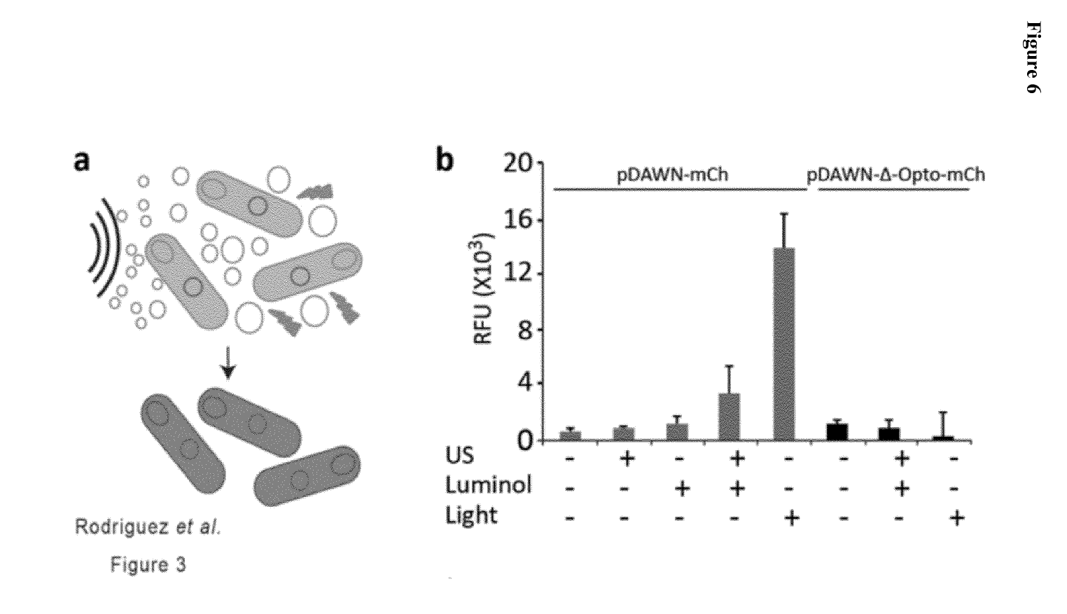

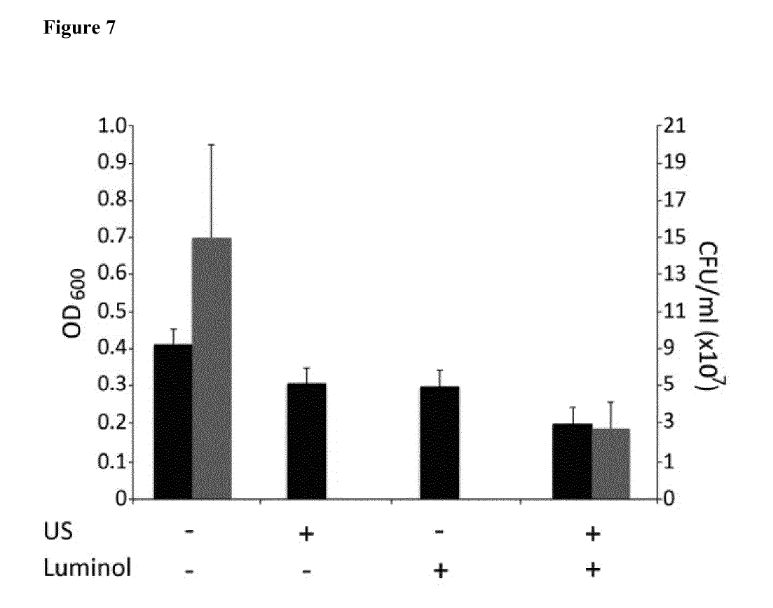

Encouraged by the sizeable light obtained in SCL we went on to test if this light can be harnessed to control a biological process in living cells (FIG. 6a). We choose to work with E. coli bacteria, which have been applied for decades for laboratory research and for the production of valuable substances. We first tested whether US stimulation affected E. coli viability in M9 minimal media using two independent measurements (optical density, OD600, and colony forming units, CFUs) (FIG. 7). We found that both indicators were reduced by ˜50% upon treatment with US and luminol (FIG. 7). Experiments with US and luminol alone showed that E. coli inactivation resulted from both physical and chemical mechanisms during US stimulation, such as free-radical attack and physical disruption of membranes, as well as the basic pH required for luminol luminescence (FIG. 7). In future studies, viability may be improved by reduced power increased frequencies and improved reaction conditions (see below) [30]. The biological process we focused on for optogenetic control was gene transcription. In particular, activation of gene transcription is a first and tightly-regulated step towards protein biosynthesis. Proteins are the major catalytic components in biological systems and valuable biotechnological products. In addition, many proteins are stable for extended periods of time (hours to days) making them ideally suited for conversion of sound into a long lasting signal. Among the available light-induced gene transcription systems, we choose the optogenetic gene regulation plasmid pDAWN [25], which contains a blue light sensitive light-oxygen-voltage (LOV) domain core, for several reasons. First, the light produced by luminol [31] spectrally overlaps with the light absorbed by LOV domains [32]. Second, this optogenetic gene regulation plasmid has a high dynamic range, and, third, the plethora of structural and functional information available on LOV domains allows tuning of system properties (e.g. sensitivity and specificity) [33]. As our first protein product we employed the red fluorescent protein mCherry [34], which is bright, stable and spectrally separated from luminol chemiluminescence. Strikingly, we found significant induction of mCherry production in E. coli cultures in response to US stimulation (˜5-fold increase compared to control experiments) (FIG. 6b). Control experiments showed (i) that cells do not respond to US alone or luminol alone with significant activation of gene transcription and (ii) that the combination of US and luminol does not induce gene activation after loss of a cysteine residue that is required for LOV domain photoactivation through cysteinyl-flavin adduct formation under moderate light conditions [32, 35]. Collectively, these results demonstrate the US stimulation can be coupled to gene transcription in living cells.

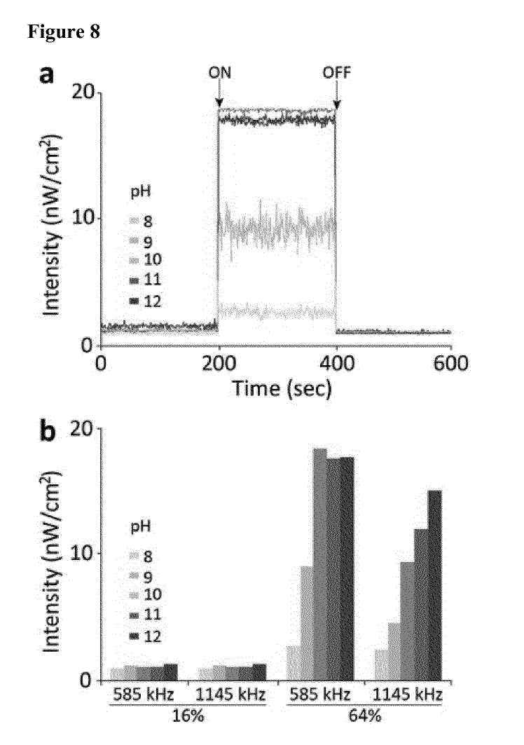

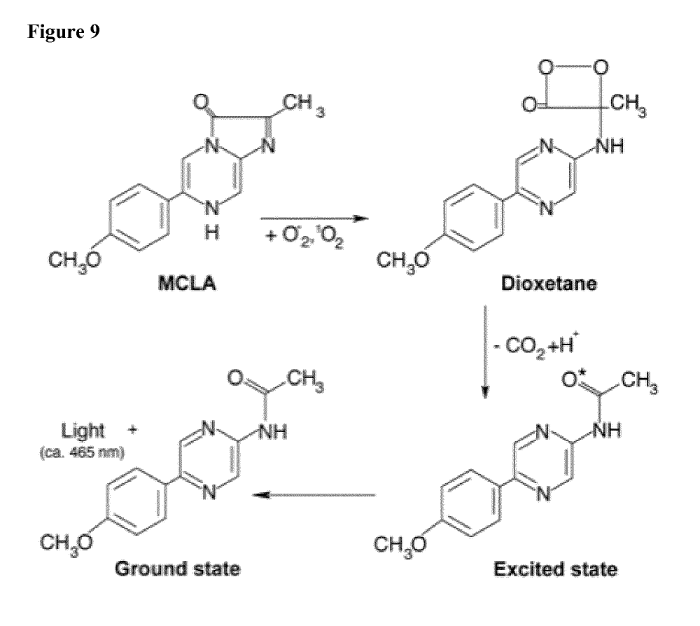

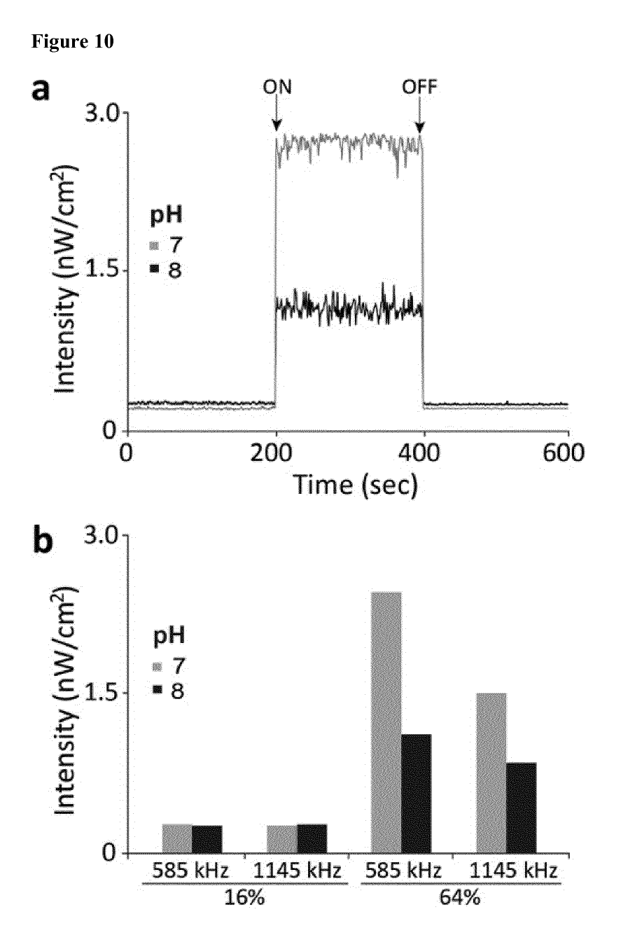

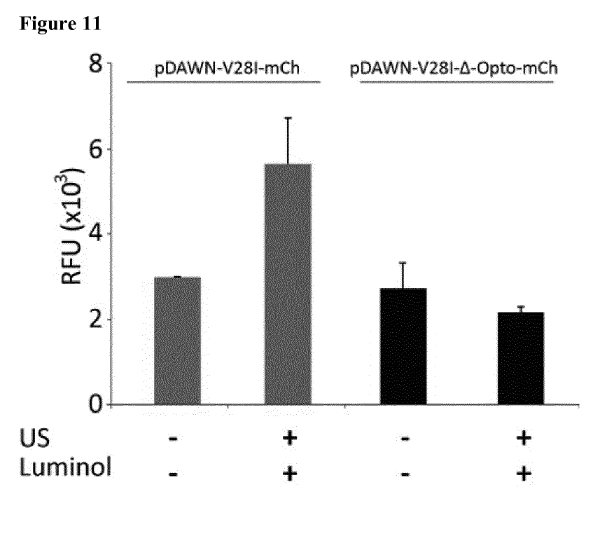

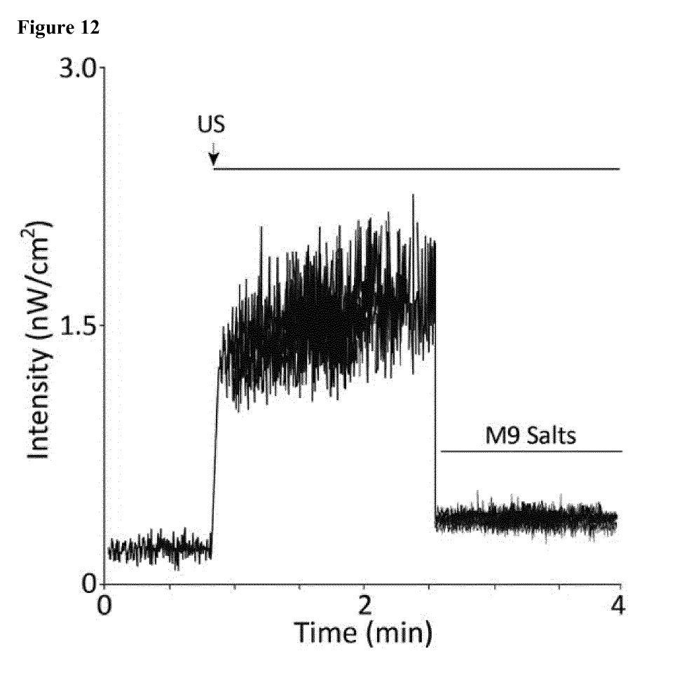

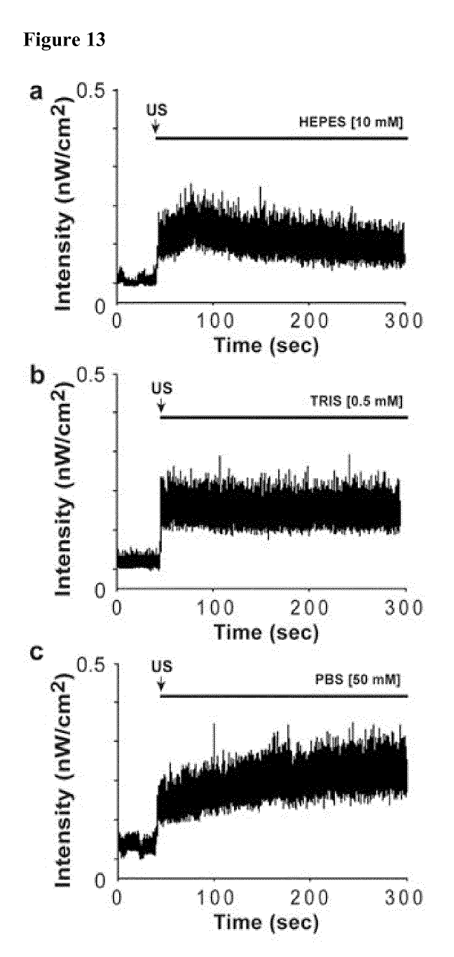

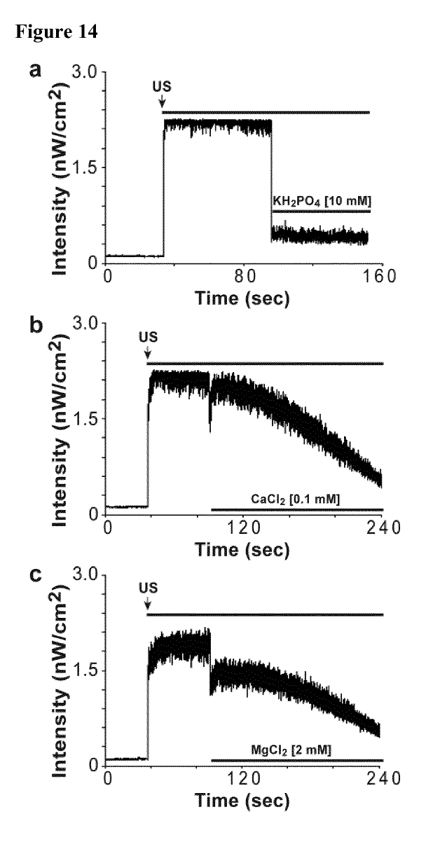

As already mentioned above, luminol chemiluminescence is pH-sensitive with optimal efficiency at basic conditions (pH≥10, FIG. 8) because hydroxyl ions catalyze an initial deprotonation step as well as excited state formation (FIG. 4). Because pH contributed to E. coli inactivation (see above), we improved the method further in two ways. First, we demonstrated a mode of operation that contained two separate chambers, one for the S(C)L reaction and one the light-sensitive process (see Example 2). Second, we showed that an alternative chemiluminescence agent, Cypridina luciferin methoxy analogue (MCLA), can generate light upon US stimulation at pH 7 (FIGS. 9 and 10) and activation of gene transcription in a dual chamber experiment (FIG. 11). In turn, MCLA-catalyzed SCL depended on solution composition, including media components, ions and buffers (FIGS. 12, 13 and 14)

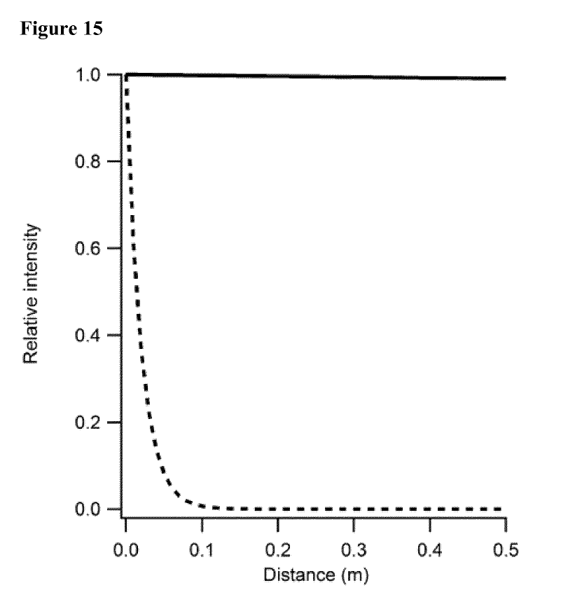

We envision that the capacity to specifically control cellular behaviors with US will open the door to new applications in acoustics and biotechnology. First, we showed that US stimulation can be coupled to gene transcription in living bacteria that are commonly used for production of biomolecules. This mode of regulation may prove especially useful in turbid media, which are penetrated readily by sound (FIG. 15 and Table 4) and in which light can now be generated ‘internally’. Second, because the vast majority of existing optogenetic methods is activated by blue light, our work can serve as a blue print for the control of these methods by US, provided that they are sufficiently light-sensitive. In particular, the regulation of disease-related behaviors of mammalian cells in tissue structures might be an interesting application for these methods (Example 2). Third, one could speculate that US generated light may also be used for energy generation in processes akin to photosynthesis, although any such process would certainly suffer from low efficiency. Finally, this approach has the potential to be harnessed for the development of novel cavitation sensors that are small and compatible with biological systems. Notably, it is currently not possible to determine ultrasonic field parameters, such as pressures, directly in biological tissues [14, 36-38]. Standard measurements are typically performed in water baths using hydrophones, which can be easily be damaged mechanically and by cavitation [39], and then followed by extrapolations using propagation models to establish the safety guidelines of US use in medicine [40, 41]. One further reason for limited understanding is that many factors affect the threshold for cavitation formation (initial nucleus size and character, pulse features, host fluid composition like content of dissolved gas, density and temperature). This context highlights the necessity to establish acoustic (cavitational) sensors on the nano- or micrometer scales, and this could be realized based on the control of a biological process (e.g. gene transcription) in living cells (e.g. live microorganisms encapsulated such that chemical equilibration with the environment is possible).

TABLE 4
Measurements of light penetration of three wavelenghts trough
E. coli cultures (OD₆₀₀ ~0.4). See Methods section for details.
One representative experiment is shown.

| | Wavelength (nm) | | |
|---|---|---|---|
| | 630 | 530 | 470 |
| Transmittance (I/I₀) | 0.29 | 0.23 | 0.31 |

### Methods

### Optogenetic Expression Plasmid

The optogenetic gene regulation plasmid pDAWN [25] contains the histidine kinase YF1, which employs a light-oxygen-voltage-sensing (LOV) domain to phosphorylate its cognate response regulator FixJ, which in turn drives gene expression from the pFixK2 promoter in a light-repressed fashion. Insertion of the lambda phage repressor cI and the lambda promoter pR in pDAWN inverts signal polarity, to confer light-activated expression of target genes introduced in a multiple-cloning site downstream of pFixK2 promoter (Table 5). mCherry [34] was a kind gift of R. Y. Tsien (University of California) and was inserted into pDAWN using polymerase chain reaction (PCR) and the BamHI and HindIII restriction enzymes to yield pDAWN-mCherry (Table 5). mCherry expression can then be detected upon light activation by red fluorescence measurements (see below).

### Site Directed Mutagenesis and Null Mutated Plasmid

A conserved cysteine-residue is required for the cysteinyl-flavin adduct formation in LOV domains [32, 35]. Thus a single amino acid substitution C62S (numbering relative to the initial valine of the photoreceptor YF1; the corresponding nucleotide substitution is g185c) was introduced into pDAWN-mCherry to yield pDAWN-ΔOPTO-mCherry, a negative control plasmid with reduced light sensitivity. Substitution was performed using site-direct mutagenesis with oligonucleotides (Table 6). All constructs used in this study were verified by sequencing of YF1, cI and the gene insert (see Table 7 for sequencing oligonucleotides).

E. coli Cultures

For starting cultures, E. coli BL21 (DE) cells transformed with pDAWN were grown overnight (225 rpm, 37° C.) in LB medium (pH 7.0; Sigma) supplemented with 50 μg/ml kanamycin (Sigma). Cells were then diluted 100-fold in fresh LB medium (pH 8.0) and growth was allowed to reach OD₆₀₀˜0.4 (7.3×10⁷ CFU/ml) under non-inducing conditions (i.e. in the dark). For US experiments, 250 ml of these cultures were centrifuged (4700 rpm, 5 min) and resuspended in 200 ml M9 minimal medium (1×M9 salts, 2 mM MgSO₄, 0.1 mM CaCl₂, 0.4% glucose, buffered with 0.05 M Tris, pH 9.0). Each culture was treated with US for 3 h. After stimulation, aliquots (50 ml) were centrifuged, resuspended (in M9 minimal media) and incubated in 96-well plates overnight under non-inducing conditions (dark) to allow mCherry maturation.

Quantification of E. coli Growth and Fluorescence Measurements

OD₆₀₀ and mCherry fluorescence emission spectra (λₑₓ=543±10 nm, λₑₘ=575 to 695 nm in 10 nm increments) were measured with a (Biotek Synergy H1) microplate reader in a black 96-well flat-bottom plates (typical volume was 200 μl). Colony forming units (CFUs) were determined by plating 100 μl of diluted cultures (dilution factors ranging between 10⁵ and 10⁷) followed by colony counting after overnight incubation (FIG. 7).

### Ultrasound Device

Experiments were conducted in the device shown in FIG. 1. The device consisted of a function generator and multi-frequency amplifier (585, 864 and or 1145 kHz; Meinhardt Ultraschalltechnik) and matched US transducer (also see main text). The transducer was fixed to the bottom of a glass reaction chamber (90 mm external diameter, 70 mm internal diameter, ˜500 ml volume). US intensities were determined as described below (also see Table 3 and FIG. 2).

Because transducer operation resulted in the heating of solutions in the chamber a cooling system was constructed. Tubing (PlasticFlex, 13 mm external diameter) was connected to the outside cylinder of the chamber. A drinking water tap was equipped with flow meter (LZT Instruments Co., measurement range 0.2-2.0 l/min) and flow regulator. The influx tube was placed in an ice bath for 24 cm of its length and 35 cm away from the chamber. Temperature was measured using a digital thermometer and maintained at 30±3° C. by adjusting the water flow rate to 1.8±2 l/min.

### Power Measurement

Total power of the bulk acoustic waves was estimated using a calorimetric method in the absence of water cooling (also see main text). A thin walled (8 mm wall thickness) aluminum chamber was constructed to fit on top of the transducer element. The chamber was filled with water as the heating material and the temperature increase in response to stimulation was measured using a digital thermometer. The total power, W, was then derived from

$$W = \frac{Δ T}{Δ t} CM$$

where ΔT is the rise in temperature, Δt is the exposure time (180 s), C is the thermal constant of pure water (4.18 J g¹ K¹) and M is the mass of the water (200 g).

Under the assumption that a directional field is produced by the transducer, the power per unit area, I, was estimated using the transducer surface area (44.1 cm²).

Pressure levels were then inferred following

P=\sqrt{1Z}

where Z is the characteristic water acoustic impedance (1.48×10⁶ kg m⁻² s⁻¹). Results are summarized in Table 3.

TABLE 3
Calorimetric method for determining US intensity at 585 kHz.
See Methods and Main Text section for details. For temperature
measurements, mean values +/− SD (three independent
experiments) are given.

| Instrument | | | | |
|---|---|---|---|---|
| setting (relative | | | | |
| power) | 16% | 32% | 48% | 64% |
| Temperature | 0.2° C. ± 0.1 | 0.7° C. ± 0.1 | 1.3° C. ± 0.2 | 2.4° C. ± 0.1 |
| change, ΔT | | | | |
| (K; in 180 s) | | | | |
| Temperature | 0.0013 | 0.0038 | 0.0074 | 0.0135 |
| change per unit | | | | |
| time, ΔT/dt | | | | |
| (K/s) | | | | |
| Total power, W | 1.0837 | 3.2511 | 6.1926 | 11.3015 |
| (W) | | | | |
| Power per unit | 0.0246 | 0.0737 | 0.1404 | 0.2562 |
| area, I (W/cm²) | | | | |
| Pressure, P | 19.0809 | 33.0267 | 45.5842 | 61.5773 |
| (kPa) | | | | |
| Pressure, | 0.19080 | 0.33026 | 0.45584 | 0.61577 |
| P (bar) | | | | |

### Light Measurements Using PMT

A dark box to house the device and a photodetection system was constructed from black polyvinyl chloride (L×W×H=47×35×43 cm) (FIG. 1). Light was measured with a PMT (H10426, Hamamatsu; 25 mm effective photocathode diameter) that was calibrated in house (see below). The PMT was placed on the side of the reaction chamber. An AD converter (USB-DT9818 with QuickDAQ software, Data Translation) was applied for data acquisition at the output offset value of 1 V. Off-line data analysis was performed in MATLAB software (TheMathWorks).

### PMT Calibration

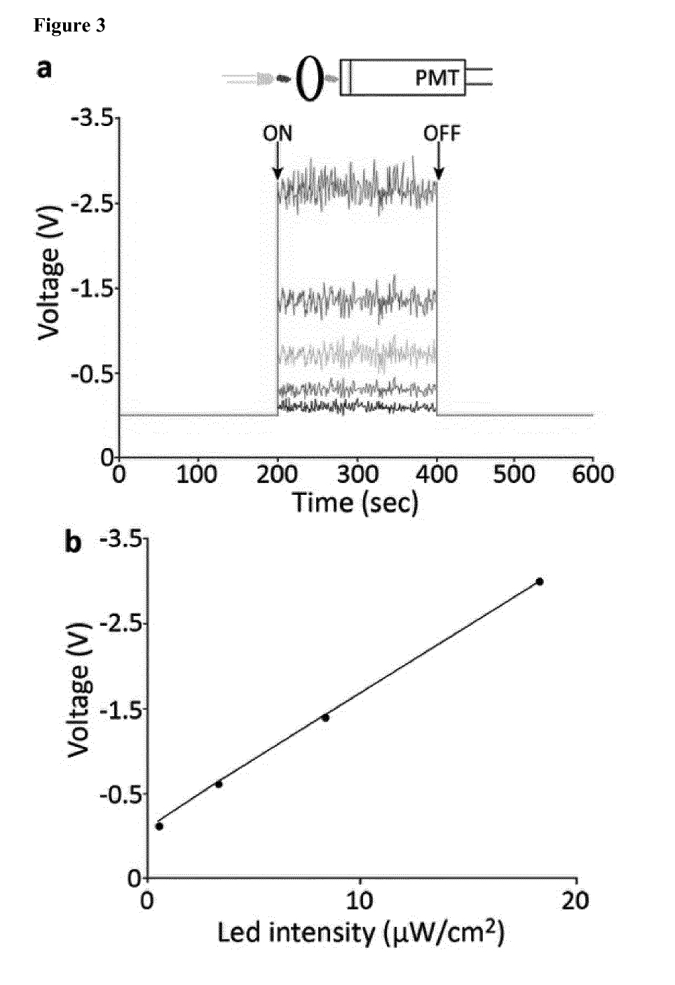

To estimate light intensity emission from our device, we characterized a small blue LED using a digital power meter (Pᵢ in W/cm²) (PM100D; Thorlabs). Next, the PMT voltage (Vᵢ) was determined for at several voltages applied to the LED using a neutral density filter (3.0). These values were then used to plot (Vᵢ) against (Pᵢ)/ND (FIG. 3).

### Attenuation of Light and Sound Waves

To illustrate the potential of US as a deeply penetrating stimulus in turbid media, attenuation of light and sound waves was modelled as a function of distance from the source (here for early log phase E. coli cultures) (FIG. 15). The relative attenuation of sound waves was described following the general equation

I(x)=e^{(-μ}^{^{S}}^{x)}

where I(x) is the relative intensity at distance x and μ^{S} denotes the sound attenuation coefficient (μ^{S}=0.0183 Np m⁻¹ for water at 0.585 MHz).

The relative attenuation of light for an early log phase E. coli culture was described using the same equation but replacing μ^{S} with the product of E. coli extinction coefficient and cell number (μ^{L}=50 Np m⁻¹ for a E. coli culture of OD₆₀₀=0.4).

In control experiments, we measured light attenuation in E. coli cultures for three wavelengths (RGB LED 5050SMD, λ˜630, 530 and 470 nm, bandwidth˜10 nm) through cultures (OD₆₀₀ 0.4; 4 cm path length). Light intensity was controlled with a dimmer and transmission was measured with a digital power meter (Table 4).

TABLE 5
DNA sequence of pDAWN, pDAWN-mCherry and pDAWN-ΔOpto-mCherry.

| Name | Sequence |
|---|---|
| pDAWN | ATCCGGATATAGTTCCTCCTTTCAGCAAAAAACCCCTCAAGACCCGTTTAGAG |
| (SEQ ID | GCCCCAAGGGGTTATGCTAGTTATTGCTCAGCGGTGGCAGCAGCCAACTCAGC |
| NO: 1) | TTCCTTTCGGGCTTTGTTAGCAGCCGGATCTCAGTGGTGGTGGTGGTGGTGCT |
| Addgene: | CGAGTGCGGCCGCAAGCTTGTCGACGGAGCTCGAATTCGGATCCGACCCATTT |
| 43796 | GCTGTCCACCAGTCATGCTAGCCATATGGCTGCCGCGCGGCACCAGGCCGCTG |
| GenBank: | CTGTGATGATGATGATGATGGCTGCTGCCCATGGTATATCTCCTTCTTAAAGT |
| JN5791211 | TAAACAAAATTATTTCTAGAGCAACCATTATCACCGCCAGAGGTAAAATAGTC |
| | AACACGCACGGTGTTACTCTAGTATATAAACGCAGAAAGGCCCACCCGAAGGT |
| | GAGCCAGTGTGACTCTAGTAGAGAGCGTTCACCGACAAACAACAGATAAAACG |
| | AAAGGCCCAGTCTTTCGACTGAGCCTTTCGTTTTATTTGATGCCTGGCTCTAG |
| | TAGCGATCTACACTAGCACTATCAGCGTTATTAAGCTACTAAAGCGTAGTTTT |
| | CGTCGTTTGCAGCGCCAAACGTCTCTTCAGGCCACTGACTAGCGATAACTTTC |
| | CCCACAACGGAACAACTCTCATTGCATGGGATCATTGGGTACTGTGGGTTTAG |
| | TGGTTGTAAAAACACCTGACCGCTATCCCTGATCAGTTTCTTGAAGGTAAACT |
| | CATCACCCCCAAGTCTGGCTATGCAGAAATCACCTGGCTCAACAGCCTGCTCA |
| | GGGTCAACGAGAATTAACATTCCGTCAGGAAAGCTCGGCTTGGAGCCTGTTGG |
| | TGCGGTCATGGAATTACCTTCAACCTCAAGCCAGAATGCAGAATCACTGGCTT |
| | TTTTGGTTGTGCTTACCCATCTCTCCGCATCACCTTTGGTAAAGGTTCTAAGC |
| | TCAGGTGAGAACATCCCTGCCTGAACATGAGAAAAAACAGGGTACTCATACTC |
| | ACTTCTAAGTGACGGCTGCATACTAACCGCTTCATACATCTCGTAGATTTCTC |
| | TGGCGATTGAAGGGCTAAATTCTTCAACGCTAACTTTGAGAATTTTTGCAAGC |
| | AATGCGGCGTTATAAGCATTTAATGCATTGATGCCATTAAATAAAGCACCAAC |
| | GCCTGACTGCCCCATCCCCATCTTGTCTGCGACAGATTCCTGGGATAAGCCAA |
| | GTTCATTTTTCTTTTTTTCATAAATTGCTTTAAGGCGACGTGCGTCCTCAAGC |
| | TGCTCTTGTGTTAATGGTTTCTTTTTTGTGCTCATCTAGTATTTCTCCTCTTT |
| | TCTAGACTCCGTTGTGATGACGCATTGGTACGCGGTATCGGGAGGTTCGAAAA |
| | TTTCGAGCGATATCTTAAGGGGGGTGCCTTACGTAGAACCCCGTAGGTCATGC |
| | CCGAGGCCGGTCCTGGATGGCGCGGCGGATACGCTTGAGCAGGTTTTCGTCGA |
| | GAAGCGGCTTCAAAACCACGTCTTTTACGCCGGCCTCGGCGGCCCGGGTCGAG |
| | ATGTTTTCGTCCGGATAGCCGGTGATCAGGATCACGGGCGTAGATCTCGATCC |
| | TCTACGCCGGACGCATCGTGGCCGGCATCACCGGCGCCACAGGTGCGGTTGCT |
| | GGCGCCTATATCGCCGACATCACCGATGGGGAAGATCGGGCTCGCCACTTCGG |
| | GCTCATGAGCGCTTGTTTCGGCGTGGGTATGGTGGCAGGCCCCGTGGCCGGGG |
| | GACTGTTGGGCGCCATCTCCTTGCATGCACCATTCCTTGCGGCGGCGGTGCTC |
| | AACGGCCTCAACCTACTACTGGGCTGCTTCCTAATGCAGGAGTCGCATAAGGG |
| | AGAGCGTCGAGATCCCGGACACCATCGAATGGTGCAAAACCTTTCGCGGTATG |
| | GCATGATAGCGCCCGGAAGAGAGTCAATTGAGGGTGGTGAATGTGGCTAGTTT |
| | TCAATCATTTGGGATACCAGGACAGCTGGAAGTCATCAAAAAAGCACTTGATC |
| | ACGTGCGAGTCGGTGTGGTAATTACAGATCCCGCACTTGAAGATAATCCTATT |
| | GTCTACGTAAATCAAGGCTTTGTTCAAATGACCGGCTACGAGACCGAGGAAAT |
| | TTTAGGAAAGAACTGTCGCTTCTTACAGGGGAAACACACAGATCCTGCAGAAG |
| | TGGACAACATCAGAACCGCTTTACAAAATAAAGAACCGGTCACCGTTCAGATC |
| | CAAAACTACAAAAAAGACGGAACGATGTTCTGGAATGAATTAAATATTGATCC |
| | AATGGAAATAGAGGATAAAACGTATTTTGTCGGTATTCAGAATGATATCACCG |
| | AGCACCAGCAGACCCAGGCGCGCCTCCAGGAACTGCAATCCGAGCTCGTCCAC |
| | GTCTCCAGGCTGAGCGCCATGGGCGAAATGGCGTCCGCGCTCGCGCACGAGCT |
| | CAACCAGCCGCTGGCGGCGATCAGCAACTACATGAAGGGCTCGCGGCGGCTGC |
| | TTGCCGGCAGCAGTGATCCGAACACACCGAAGGTCGAAAGCGCCCTGGACCGC |
| | GCCGCCGAGCAGGCGCTGCGCGCCGGCCAGATCATCCGGCGCCTGCGCGACTT |
| | CGTTGCCCGCGGCGAATCGGAGAAGCGGGTCGAGAGTCTCTCCAAGCTGATCG |
| | AGGAGGCCGGCGCGCTCGGGCTTGCCGGCGCGCGCGAGCAGAACGTGCAGCTC |
| | CGCTTCAGTCTCGATCCGGGCGCCGATCTCGTTCTCGCCGACCGGGTGCAGAT |
| | CCAGCAGGTCCTGGTCAACCTGTTCCGCAACGCGCTGGAAGCGATGGCTCAGT |
| | CGCAGCGACGCGAGCTCGTCGTCACCAACACCCCCGCCGCCGACGACATGATC |
| | GAGGTCGAAGTGTCCGACACCGGCAGCGGTTTCCAGGACGACGTCATTCCGAA |
| | CCTGTTTCAGACTTTCTTCACCACCAAGGACACCGGCATGGGCGTGGGACTGT |
| | CCATCAGCCGCTCGATCATCGAAGCTCACGGCGGGCGCATGTGGGCCGAGAGC |
| | AACGCATCGGGCGGGGCGACCTTCCGCTTCACCCTCCCGGCAGCCGACGAGAT |
| | GATAGGAGGTCTAGCATGACGACCAAGGGACATATCTACGTCATCGACGACGA |
| | CGCGGCGATGCGGGATTCGCTGAATTTCCTGCTGGATTCTGCCGGCTTCGGCG |
| | TCACGCTGTTTGACGACGCGCAAGCCTTTCTCGACGCCCTGCCGGGTCTCTCC |
| | TTCGGCTGCGTCGTCTCCGACGTGCGCATGCCGGGCCTTGACGGCATCGAGCT |
| | GTTGAAGCGGATGAAGGCGCAGCAAAGCCCCTTTCCGATCCTCATCATGACCG |
| | GTCACGGCGACGTGCCGCTCGCGGTCGAGGCGATGAAGTTAGGGGCGGTGGAC |
| | TTTCTGGAAAAGCCTTTCGAGGACGACCGCCTCACCGCCATGATCGAATCGGC |
| | GATCCGCCAGGCCGAGCCGGCCGCCAAGAGCGAGGCCGTCGCGCAGGATATCG |
| | CCGCCCGCGTCGCCTCGTTGAGCCCCAGGGAGCGCCAGGTCATGGAAGGGCTG |
| | ATCGCCGGCCTTTCCAACAAGCTGATCGCCCGCGAGTACGACATCAGCCCGCG |
| | CACCATCGAGGTGTATCGGGCCAACGTCATGACCAAGATGCAGGCCAACAGCC |
| | TTTCGGAGCTGGTTCGCCTCGCGATGCGCGCCGGCATGCTCAACGATTGACAA |
| | TTGATGTAAGTTAGCTCACTCATTAGGCACCGGGATCTCGACCGATGCCCTTG |
| | AGAGCCTTCAACCCAGTCAGCTCCTTCCGGTGGGCGCGGGGCATGACTATCGT |
| | CGCCGCACTTATGACTGTCTTCTTTATCATGCAACTCGTAGGACAGGTGCCGG |
| | CAGCGCTCTGGGTCATTTTCGGCGAGGACCGCTTTCGCTGGAGCGCGACGATG |
| | ATCGGCCTGTCGCTTGCGGTATTCGGAATCTTGCACGCCCTCGCTCAAGCCTT |
| | CGTCACTGGTCCCGCCACCAAACGTTTCGGCGAGAAGCAGGCCATTATCGCCG |
| | GCATGGCGGCCCCACGGGTGCGCATGATCGTGCTCCTGTCGTTGAGGACCCGG |
| | CTAGGCTGGCGGGGTTGCCTTACTGGTTAGCAGAATGAATCACCGATACGCGA |
| | GCGAACGTGAAGCGACTGCTGCTGCAAAACGTCTGCGACCTGAGCAACAACAT |
| | GAATGGTCTTCGGTTTCCGTGTTTCGTAAAGTCTGGAAACGCGGAAGTCAGCG |
| | CCCTGCACCATTATGTTCCGGATCTGCATCGCAGGATGCTGCTGGCTACCCTG |
| | TGGAACACCTACATCTGTATTAACGAAGCGCTGGCATTGACCCTGAGTGATTT |
| | TTCTCTGGTCCCGCCGCATCCATACCGCCAGTTGTTTACCCTCACAACGTTCC |
| | AGTAACCGGGCATGTTCATCATCAGTAACCCGTATCGTGAGCATCCTCTCTCG |
| | TTTCATCGGTATCATTACCCCCATGAACAGAAATCCCCCTTACACGGAGGCAT |
| | CAGTGACCAAACAGGAAAAAACCGCCCTTAACATGGCCCGCTTTATCAGAAGC |
| | CAGACATTAACGCTTCTGGAGAAACTCAACGAGCTGGACGCGGATGAACAGGC |
| | AGACATCTGTGAATCGCTTCACGACCACGCTGATGAGCTTTACCGCAGCTGCC |
| | TCGCGCGTTTCGGTGATGACGGTGAAAACCTCTGACACATGCAGCTCCCGGAG |
| | ACGGTCACAGCTTGTCTGTAAGCGGATGCCGGGAGCAGACAAGCCCGTCAGGG |
| | CGCGTCAGCGGGTGTTGGCGGGTGTCGGGGCGCAGCCATGACCCAGTCACGTA |
| | GCGATAGCGGAGTGTATACTGGCTTAACTATGCGGCATCAGAGCAGATTGTAC |
| | TGAGAGTGCACCATATATGCGGTGTGAAATACCGCACAGATGCGTAAGGAGAA |
| | AATACCGCATCAGGCGCTCTTCCGCTTCCTCGCTCACTGACTCGCTGCGCTCG |
| | GTCGTTCGGCTGCGGCGAGCGGTATCAGCTCACTCAAAGGCGGTAATACGGTT |
| | ATCCACAGAATCAGGGGATAACGCAGGAAAGAACATGTGAGCAAAAGGCCAGC |
| | AAAAGGCCAGGAACCGTAAAAAGGCCGCGTTGCTGGCGTTTTTCCATAGGCTC |
| | CGCCCCCCTGACGAGCATCACAAAAATCGACGCTCAAGTCAGAGGTGGCGAAA |
| | CCCGACAGGACTATAAAGATACCAGGCGTTTCCCCCTGGAAGCTCCCTCGTGC |
| | GCTCTCCTGTTCCGACCCTGCCGCTTACCGGATACCTGTCCGCCTTTCTCCCT |
| | TCGGGAAGCGTGGCGCTTTCTCATAGCTCACGCTGTAGGTATCTCAGTTCGGT |
| | GTAGGTCGTTCGCTCCAAGCTGGGCTGTGTGCACGAACCCCCCGTTCAGCCCG |
| | ACCGCTGCGCCTTATCCGGTAACTATCGTCTTGAGTCCAACCCGGTAAGACAC |
| | GACTTATCGCCACTGGCAGCAGCCACTGGTAACAGGATTAGCAGAGCGAGGTA |
| | TGTAGGCGGTGCTACAGAGTTCTTGAAGTGGTGGCCTAACTACGGCTACACTA |
| | GAAGGACAGTATTTGGTATCTGCGCTCTGCTGAAGCCAGTTACCTTCGGAAAA |
| | AGAGTTGGTAGCTCTTGATCCGGCAAACAAACCACCGCTGGTAGCGGTGGTTT |
| | TTTTGTTTGCAAGCAGCAGATTACGCGCAGAAAAAAAGGATCTCAAGAAGATC |
| | CTTTGATCTTTTCTACGGGGTCTGACGCTCAGTGGAACGAAAACTCACGTTAA |
| | GGGATTTTGGTCATGAACAATAAAACTGTCTGCTTACATAAACAGTAATACAA |
| | GGGGTGTTATGAGCCATATTCAACGGGAAACGTCTTGCTCTAGGCCGCGATTA |
| | AATTCCAACATGGATGCTGATTTATATGGGTATAAATGGGCTCGCGATAATGT |
| | CGGGCAATCAGGTGCGACAATCTATCGATTGTATGGGAAGCCCGATGCGCCAG |
| | AGTTGTTTCTGAAACATGGCAAAGGTAGCGTTGCCAATGATGTTACAGATGAG |
| | ATGGTCAGACTAAACTGGCTGACGGAATTTATGCCTCTTCCGACCATCAAGCA |
| | TTTTATCCGTACTCCTGATGATGCATGGTTACTCACCACTGCGATCCCCGGGA |
| | AAACAGCATTCCAGGTATTAGAAGAATATCCTGATTCAGGTGAAAATATTGTT |
| | GATGCGCTGGCAGTGTTCCTGCGCCGGTTGCATTCGATTCCTGTTTGTAATTG |
| | TCCTTTTAACAGCGATCGCGTATTTCGTCTCGCTCAGGCGCAATCACGAATGA |
| | ATAACGGTTTGGTTGATGCGAGTGATTTTGATGACGAGCGTAATGGCTGGCCT |
| | GTTGAACAAGTCTGGAAAGAAATGCATAAACTTTTGCCATTCTCACCGGATTC |
| | AGTCGTCACTCATGGTGATTTCTCACTTGATAACCTTATTTTTGACGAGGGGA |
| | AATTAATAGGTTGTATTGATGTTGGACGAGTCGGAATCGCAGACCGATACCAG |
| | GATCTTGCCATCCTATGGAACTGCCTCGGTGAGTTTTCTCCTTCATTACAGAA |
| | ACGGCTTTTTCAAAAATATGGTATTGATAATCCTGATATGAATAAATTGCAGT |
| | TTCATTTGATGCTCGATGAGTTTTTCTAAGAATTAATTCATGAGCGGATACAT |
| | ATTTGAATGTATTTAGAAAAATAAACAAATAGGGGTTCCGCGCACATTTCCCC |
| | GAAAAGTGCCACCTGAAATTGTAAACGTTAATATTTTGTTAAAATTCGCGTTA |
| | AATTTTTGTTAAATCAGCTCATTTTTTAACCAATAGGCCGAAATCGGCAAAAT |
| | CCCTTATAAATCAAAAGAATAGACCGAGATAGGGTTGAGTGTTGTTCCAGTTT |
| | GGAACAAGAGTCCACTATTAAAGAACGTGGACTCCAACGTCAAAGGGCGAAAA |
| | ACCGTCTATCAGGGCGATGGCCCACTACGTGAACCATCACCCTAATCAAGTTT |
| | TTTGGGGTCGAGGTGCCGTAAAGCACTAAATCGGAACCCTAAAGGGAGCCCCC |
| | GATTTAGAGCTTGACGGGGAAAGCCGGCGAACGTGGCGAGAAAGGAAGGGAAG |
| | AAAGCGAAAGGAGCGGGCGCTAGGGCGCTGGCAAGTGTAGCGGTCACGCTGCG |
| | CGTAACCACCACACCCGCCGCGCTTAATGCGCCGCTACAGGGCGCGTCCCATT |
| | CGCCA |
| pDAWN- | ...GTGGCTAGTTTTCAATCATTTGGGATACCAGGACAGCTGGAAGTCATCAAAA |
| ΔOpto- | AAGCACTTGATCACGTGCGAGTCGGTGTGGTAATTACAGATCCCGCACTTGAA |
| mCherry | GATAATCCTATTGTCTACGTAAATCAAGGCTTTGTTCAAATGACCGGCTACGA |
| (SEQ ID | GACCGAGGAAATTTTAGGAAAGAACTCTCGCTTCTTACAGGGGAAACACACAG |
| NO: 2) | ATCCTGCAGAAGTGGACAACATCAGAACCGCTTTACAAAATAAAGAACCGGTC |
| | ACCGTTCAGATCCAAAACTACAAAAAAGACGGAACGATGTTCTGGAATGAATT |
| | AAATATTGATCCAATGGAAATAGAGGATAAAACGTATTTTGTCGGAATTCAGA |
| | ATGATATCACCGAGCACCAGCAGACCCAGGCGCGCCTCCAGGAACTGCAATCC |
| | GAGCTCGTCCACGTCTCCAGGCTGAGCGCCATGGGCGAAATGGCGTCCGCGCT |
| | CGCGCACGAGCTCAACCAGCCGCTGGCGGCGATCAGCAACTACATGAAGGGCT |
| | CGCGGCGGCTGCTTGCCGGCAGCAGTGATCCGAACACACCGAAGGTCGAAAGC |
| | GCCCTGGACCGCGCCGCCGAGCAGGCGCTGCGCGCCGGCCAGATCATCCGGCG |
| | CCTGCGCGACTTCGTTGCCCGCGGCGAATCGGAGAAGCGGGTCGAGAGTCTCT |
| | CCAAGCTGATCGAGGAGGCCGGCGCGCTCGGGCTTGCCGGCGCGCGCGAGCAG |
| | AACGTGCAGCTCCGCTTCAGTCTCGATCCGGGCGCCGATCTCGTTCTCGCCGA |
| | CCGGGTGCAGATCCAGCAGGTCCTGGTCAACCTGTTCCGCAACGCGCTGGAAG |
| | CGATGGCTCAGTCGCAGCGACGCGAGCTCGTCGTCACCAACACCCCCGCCGCC |
| | GACGACATGATCGAGGTCGAAGTGTCCGACACCGGCAGCGGTTTCCAGGACGA |
| | CGTCATTCCGAACCTGTTTCAGACTTTCTTCACCACCAAGGACACCGGCATGG |
| | GCGTGGGACTGTCCATCAGCCGCTCGATCATCGAAGCTCACGGCGGGCGCATG |
| | TGGGCCGAGAGCAACGCATCGGGCGGGGCGACCTTCCGCTTCACCCTCCCGGC |
| | AGCCGACGAGATGATAGGAGGTCTAGCATGACGACCAAGGGACATATCTACGT |
| | CATCGACGACGACGCGGCGATGCGGGATTCGCTGAATTTCCTGCTGGATTCTG |
| | CCGGCTTCGGCGTCACGCTGTTTGACGACGCGCAAGCCTTTCTCGACGCCCTG |
| | CCGGGTCTCTCCTTCGGCTGCGTCGTCTCCGACGTGCGCATGCCGGGCCTTGA |
| | CGGCATCGAGCTGTTGAAGCGGATGAAGGCGCAGCAAAGCCCCTTTCCGATCC |
| | TCATCATGACCGGTCACGGCGACGTGCCGC... |

TABLE 6
Oligonucleotides for
introducing point substitutions.

| Name | Sequence |
|---|---|
| YF1_C62S_F | GAAATTTTAGGAAAGAACTCTCGCTT |
| (SEQ ID NO: 3) | CTTACAGGGGAAAC |
| YF1_C62S_R | GTTTCCCCTGTAAGAAGCGAGAGTTC |
| (SEQ ID NO: 4) | TTTCCTAAAATTTC |

TABLE 7
Oligonucleotides used for sequencing.

| Name | Sequence |
|---|---|
| T7_terminal_R | GCTAGTTATTGCTCAGCGG |
| (SEQ ID NO: 5) | |
| Xpress_R | CCAGTCATGCTAGCCATA |
| (SEQ ID NO: 6) | |
| YF1_seq_1F | gtggctagttttcaatca |
| (SEQ ID NO: 7) | |
| cI_middle seq_F | CTCTGGCGATTGAAGGG |
| (SEQ ID NO: 8) | |
| YF1_middle_seq_F | AACCTGTTCCGCAACGC |
| (SEQ ID NO: 9) | |

### Example 2

This Examples applies US to control the signaling/morphology of mammalian cells in artificial tissues.

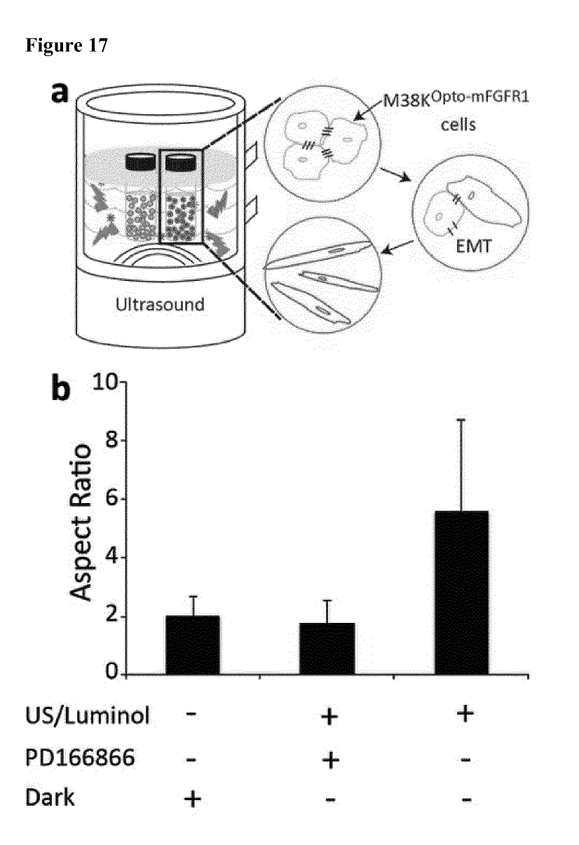

We used a cell line originally derived from a malignant pleural mesothelioma (M38K) that is naturally responsive to growth factors [42]. We extended this cell line, now called M38K^{Opto-mFGFR1}, by expressing Opto-mFGFR1, a modified fibroblast growth factor receptor that is exclusively activated by blue light through the light-oxygen-voltage-sensing (LOV) domain of aureochrome 1 from Vaucheria frigida [21, 22]. First, we tested the performance of SCL to activate intracellular signaling in the described mammalian cell line in the two chambers configuration setup (see Methods below). Data analysis shows that luminol SCL induced epithelial-mesenchymal transition (EMT)-like morphological changes in M38K^{Opto-mFGFR1} cells (FIG. 17a). In a control experiment, we pre-treated the cells with PD166866, a selective FGFR1 kinase inhibitor, and we found that the effect of luminol SCL was abolished by the drug, as expected for a specific response through the Opto-mFGFR1 receptors (FIG. 17b).

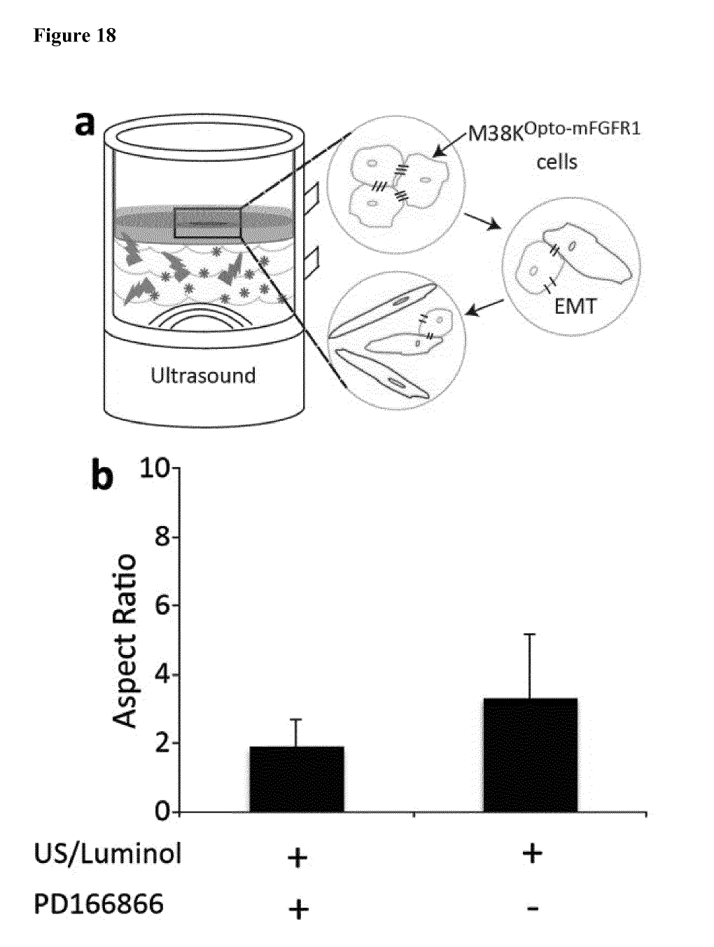

Next, we analyzed whether SCL could also activate the same mechanism in M38K^{Opto-mFGFR1} cells through a mimic tissue [43] (porcine gelatin of 2 cm thickness). Also in this case, US resulted in an increased aspect ratio indicative of induced epithelial-mesenchymal transition (EMT)-like morphological changes of (FIG. 18).

Collectively, these experiments demonstrate controlled changes in cell behavior induced by US trough luminol SCL in cell monolayers and also through mimic biological tissues.

### Methods

### M38KOpto-mFGFR1 Cells

M38KOpto-mFGFR1 cell line was maintained at 37° C. in RPMI1640 media supplemented with 10% FBS, 100 U/ml penicillin and 0.1 mg/ml streptomycin. For experimentation, 10^{5 M38KOpto-mFGFR1} cells were seeded in petri dishes (35 mm) containing glass beads (600-800 μm, ESSKA GmbH, Germany, previously coated with poly-L-Ornithine; dilution 1:25 from Sigma, Austria at 4° C. overnight) or directly into the petri dish containing one coverslip of 22 mm. After 24 h of incubation, cells were transferred into a glass vial (1.5 cm diameter; SUPELCO, Sigma Aldrich, USA) containing 2 ml RPMI1640 media, and placed immediately into the US reactor using a floating foam tubes holder (Invitrogen). The US reactor was filled with 250 ml of luminol solution (20 mM Luminol, 100 μM EDTA, 100 μM Na₂CO₃, 10 mM H₂O₂; all reagents were purchased from Sigma, Austria). For control experiments, PD166866 (Pfizer Global Research and Development, New London, Conn.) was added to a final concentration of 10 μM for 1 h before US stimulation (585 kHz, 64% amplitude, 3 h). After additional 24 h, cells were photographed on the Nikon Ti300 microscope. For quantification of cell morphology, all cell perimeters in randomly selected sections of phase contrast or fluorescent images were traced and aspect ratios (defined as length of major axis divided by length of minor axis of a fitted ellipse) calculated with ImageJ-64 software. Individual values contributed to each average. Automated analysis yielded comparable results.

### Tissue-Mimicking Phantom Fabrication

A gelatin phantom was used in this study. The phantom was composed of 92.5% de-ionized water and 1.5 g/mL porcine gelatin (Sigma-Aldrich) [43]. The ingredients were mixed with a stir bar over a stir plate and simultaneously heated to 40-45° C. Once the liquid phantom cooled down to 30° C., it was poured into the US reactor as a mold covered with commercial plastic film packaging and refrigerated for 24 h. In this configuration, M38K^{Opto-mFGFR1} cells were seeded in a coverslip, and placed on top of the porcine gelatin, embedded in the described RPMI1640 media, while luminol solution was below the mimic tissue.

### REFERENCES

1. Mulvana, H., S. Cochran, and M. Hill, Ultrasound assisted particle and cell manipulation on-chip. Adv Drug Deliv Rev, 2013. 65(11-12): p. 1600-10.
2. Ding, X., et al., On-chip manipulation of single microparticles, cells, and organisms using surface acoustic waves. Proc Natl Acad Sci USA, 2012. 109(28): p. 11105-9.
3. Wu, G., et al., Remotely triggered liposome release by near-infrared light absorption via hollow gold nanoshells. J Am Chem Soc, 2008. 130(26): p. 8175-7.
4. Mason, T. J., Sonochemistry and sonoprocessing: the link, the trends and (probably) the future. Ultrason Sonochem, 2003. 10(4-5): p. 175-9.
5. Ferrara, K. W., M. A. Borden, and H. Zhang, Lipid-shelled vehicles: engineering for ultrasound molecular imaging and drug delivery. Acc Chem Res, 2009. 42(7): p. 881-92.
6. Bang, J. H. and K. S. Suslick, Applications of ultrasound to the synthesis of nanostructured materials. Adv Mater, 2010. 22(10): p. 1039-59.
7. Drinkwater, B. W. and P. D. Wilcox, Ultrasonic arrays for non-destructive evaluation: A review. NDT & E INTERNATIONAL, 2006. 39(7): p. 525-541.
8. Fenster, A. and D. B. Downey, Three-dimensional ultrasound imaging. Annu Rev Biomed Eng, 2000. 2: p. 457-75.
9. Warden, S. J., et al., Low-intensity pulsed ultrasound stimulates a bone-forming response in UMR-106 cells. Biochem Biophys Res Commun, 2001. 286(3): p. 443-50.
10. Vogel, V. and M. Sheetz, Local force and geometry sensing regulate cell functions. Nat Rev Mol Cell Biol, 2006. 7(4): p. 265-75.
11. Steel, K. P. and C. J. Kros, A genetic approach to understanding auditory function. Nat Genet, 2001. 27(2): p. 143-9.
12. Raphael, Y. and R. A. Altschuler, Structure and innervation of the cochlea. Brain Res Bull, 2003. 60(5-6): p. 397-422.
13. Holland, K. C. and E. R. Apfel, Thresholds for transient cavitation produced by pulsed ultrasound in a controlled nuclei environment. The Journal of Acoustical Society of America, 1990. 88(5): p. 2059-2069.
14. Apfel, R., Acoustic cavitation: a possible consequence of biomedical uses of ultrasound. The British Journal of Cancer, 1982. 5: p. 140-146.
15. Duck, F. A., Medical and non-medical protection standards for ultrasound and infrasound. Prog Biophys Mol Biol, 2007. 93(1-3): p. 176-91.
16. Fernandez Rivas, D., et al., Sonoluminescence and sonochemiluminescence from a microreactor. Ultrason Sonochem, 2012. 19(6): p. 1252-9.
17. Brotchie, A., F. Grieser, and M. Ashokkumar, Effect of power and frequency on bubble-size distributions in acoustic cavitation. Phys Rev Lett, 2009. 102(8): p. 084302.
18. Putterman, S., Sonoluminescence: Sound into Light. Scientific American, 1995. 272(2): p. 46-51.
19. Muller, K. and W. Weber, Optogenetic tools for mammalian systems. Mol Biosyst, 2013. 9(4): p. 596-608.
20. Szobota, S. and E. Y. Isacoff, Optical control of neuronal activity. Annu Rev Biophys, 2010. 39: p. 329-48.
21. Grusch, M., et al., Spatio-temporally precise activation of engineered receptor tyrosine kinases by light. EMBO J, 2014. 33(15): p. 1713-26.
22. Ingles-Prieto, A., et al., Light-assisted small-molecules screening against protein kinases. Nature Chemical Biology, 2015: p. 1-3.
23. Kikuchi, T. and T. Uchida, Calorimetric method for measuring high ultrasonic power using water as a heating material. Journal of Physics: Conference Series, 2011. 279(1): p. 1-5.
24. Berndt, A., et al., Bi-stable neural state switches. Nat Neurosci, 2009. 12(2): p. 229-34.
25. Ohlendorf, R., et al., From dusk till dawn: one-plasmid systems for light-regulated gene expression. J Mol Biol, 2012. 416(4): p. 534-42.
26. Janovjak, H., et al., A light-gated, potassium-selective glutamate receptor for the optical inhibition of neuronal firing. Nat Neurosci, 2010. 13(8): p. 1027-1032.
27. Cao, H., et al., Spatial distribution of sonoluminescence and sonochemiluminescence generated by cavitation bubbles in 1.2 MHz focused ultrasound field. Ultrason Sonochem, 2012. 19(2): p. 257-63.
28. Hatanaka, S.-i., et al., Single-Bubble Sonochemiluminescence in Aqueous Luminol Solutions. J Am Chem Soc, 2002. 124: p. 10250-10251.
29. Lee, H. B. and P. K. Choi, Acoustic power dependences of sonoluminescence and bubble dynamics. Ultrason Sonochem, 2014. 21(6): p. 2037-43.
30. Gao, S., et al., Inactivation of bacteria and yeast using high-frequency ultrasound treatment. Water Res, 2014. 60: p. 93-104.
31. Taylor, K. J. and P. D. Jarman, Spectrum and lifetime of the acoustically and chemically induced emission of light from luminol. J. Am. Chem. Soc., 1971. 93(1): p. 257-258.
32. Losi, A. and W. Gartner, Old chromophores, new photoactivation paradigms, trendy applications: flavins in blue light-sensing photoreceptors. Photochem Photobiol, 2011. 87(3): p. 491-510.
33. Moglich, A. and K. Moffat, Engineered photoreceptors as novel optogenetic tools. Photochem Photobiol Sci, 2010. 9(10): p. 1286-300.
34. Shaner, N. C., et al., Improved monomeric red, orange and yellow fluorescent proteins derived from Discosoma sp. red fluorescent protein. Nat. Biotechnol., 2004. 22(12): p. 1567-72.
35. Alexandre, M. T., et al., Primary reactions of the LOV2 domain of phototropin studied with ultrafast mid-infrared spectroscopy and quantum chemistry. Biophys J, 2009. 97(1): p. 227-37.
36. Miller, D. L., Overview of experimental studies of biological effects of medical ultrasound caused by gas body activation and inertial cavitation. Prog Biophys Mol Biol, 2007. 93(1-3): p. 314-30.
37. Holland, K. C., et al., Direct evidence of cavitation in vivo from diagnostic ultrasound. Ultrasound Med Biol, 1996. 22(7): p. 917-925.
38. Kieler, H., Epidemiological studies on adverse effects of prenatal ultrasound—which are the challenges? Prog Biophys Mol Biol, 2007. 93(1-3): p. 301-8.
39. Leighton, T. G., Rapporteur report: Mechanisms and interactions. Prog Biophys Mol Biol, 2007. 93(1-3): p. 280-94.
40. ter Haar, G., et al., The safe use of ultrasound in medical diagnosis. 3rd ed. 2012, United Kingdom: The British Institute of Radiology. 163.
41. Levenback, B. J., C. M. Sehgal, and A. K. Wood, Modeling of thermal effects in antivascular ultrasound therapy. J Acoust Soc Am, 2012. 131(1): p. 540-9.
42. Kahlos, K., et al., Manganese Superoxide Dismutase in Healthy Human Pleural Mesothelium and in Malignant Pleural Mesothelioma. Am. J. Respir. Cell. Mol. Biol., 1998. 18: p. 570-580.
43. Kim, J., et al., Phantom evaluation of stacked-type dual-frequency 1-3 composite transducers: A feasibility study on intracavitary acoustic angiography. Ultrasonics, 2015. 63: p. 7-15.

1. A method of regulating at least one cellular process in one or more cells, comprising (a) introducing into the one or more cells a construct comprising a regulatory transgene capable of regulating the at least one cellular process operably linked to a light-inducible control sequence and (b) inducing the expression of the regulatory transgene using sonoluminescence (SL) or sonochemiluminescence (SCL) by exposing the one or more cells to ultrasound.

2. A method according to claim 1, wherein the light-inducible control sequence is pDAWN (SEQ ID NO: 1) or a variant thereof.

3. A method of regulating at least one cellular process in one or more cells, comprising (a) contacting the one or more cells with one or more light-sensitive proteins capable of regulating the at least one cellular process and (b) regulating the function of the one or more proteins using sonoluminescence (SL) or sonochemiluminescence (SCL) by exposing the one or more cells to ultrasound.

4. A method according to claim 3, wherein the method comprises in step (a) expressing the one or more light-sensitive proteins in the one or more cells.

5. A method according to claim 3 or 4, wherein the one or more light-sensitive proteins comprise a light-sensitive domain selected from phytochromes, LOV domains, BLUF domains, cryptochromes, opsins, photoactive yellow proteins, UV-B responsive proteins, vitamin b12 photosensors, and fluorescent proteins.

6. A method according to any one of the preceding claims, wherein the at least one cellular process comprises at least one of (i) expression and/or transcription of a target gene, (ii) function of a target protein, (iii) cellular morphology and (iv) cellular signalling.

7. A method according to claim 6, wherein the method increases expression and/or transcription of the target gene and/or increases the function of the target protein in the one or more cells.

8. A method according to claim 7, wherein the target gene is the same as the regulatory transgene.

9. A method according to claim 7, wherein the target gene encodes the target protein.

10. A method according to any one of claims 7 to 9, wherein a decreased expression and/or transcription of the target gene and/or a decreased function of the target protein in the one or more cells is associated with a disease or disorder.

11. A method according to claim 6, wherein the method decreases expression and/or transcription of the target gene and/or decreases the function of the target protein in the one or more cells.

12. A method according to claim 11, wherein the target gene encodes the target protein.

13. A method according to claim 11 or 12, wherein an increased expression and/or transcription of the target gene and/or an increased function of the target protein in the one or more cells is associated with a disease or disorder.

14. A method according to any one of claims 6 to 12, wherein the cellular morphology is neoplasia.

15. A method according to any one of claims 6 to 14, wherein expression of the method increases or decreases cellular signalling.

16. A method according to claim 15, wherein a decreased or an increased cellular signalling in the one or more cells is associated with a disease or disorder.

17. A method according to any one of claims 10, 13 and 16, wherein the disease or disorder is a (a) neoplasm or (b) a respiratory, cardiovascular, gastroenterological, skin, musculoskeletal, neurological, ophthalmological, genitourinary, immune system or inflammatory disease or disorder.

18. A method according to claim 17, wherein the neoplasm is cancer.

19. A method according to any one of the preceding claims, wherein the method further comprises contacting the one or more cells with a chemiluminescent agent and inducing the expression of the regulatory transgene or regulating the function of the one or more light-sensitive proteins using sonochemiluminescence (SCL) by exposing the one or more cells to ultrasound.

20. A method according to claim 19, wherein the chemiluminescent agent is a luminol, a luciferin, a Cyridina luciferin methoxy analogue (MCLA), a fluorescent solute, an acridinium salt or an oxalate.

21. A method according to any one of the preceding claims, wherein the one or more cells are exposed to ultrasound at a frequency of from about 25 kHz to about 40 MHz.

22. A method according to any one of the preceding claims, wherein the one or more cells are exposed to ultrasound at a power per unit area of from about 0.01 to about 500 W/cm².

23. A method according to any one of the preceding claims, wherein the one or more cells are exposed to ultrasound under conditions which result in a pressure of from about 10 kPa to about 3 MPa.

24. A method according to any one of the preceding claims, wherein the one or more cell are in vitro.

25. A method according to any one of claims 1 to 24, wherein the one or more cells are present in a subject.

26. A method according to any one of the preceding claims, wherein the one or more cells are human.

27. A construct comprising a regulatory transgene capable of regulating at least one cellular process operably linked to a light-inducible control sequence for use in a method of regulating the at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound.

28. A combination of (a) a construct comprising a regulatory transgene capable of regulating at least one cellular process operably linked to a light-inducible control sequence and (b) and an ultrasound device for use in a method of regulating the at least one cellular process in one or more cells in a subject, wherein the method comprises introducing the construct into the one or more cells and inducing the expression of the regulatory transgene using sonoluminescence (SL) or sonochemiluminescence (SCL) by exposing the one or more cells to ultrasound using the device.

29. One or more light-sensitive proteins capable of regulating at least one cellular process or one or more constructs encoding the one or more light-sensitive proteins for use in a method of regulating the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound.

30. A combination of (a) one or more light-sensitive proteins capable of regulating at least one cellular process or one or more constructs encoding the one or more light-sensitive proteins (b) and an ultrasound device for use in a method of regulating the at least one cellular process in one or more cells in a subject, wherein the method comprises contacting the one or more proteins with the one or more cells or expressing the one or more constructs in the one or more cells and regulating the function of the one or more proteins using sonoluminescence (SL) or sonochemiluminescence (SCL) by exposing the one or more cells to ultrasound.

31. A kit for of regulating at least one cellular process in one or more cells comprising (a) a construct comprising a regulatory transgene capable of regulating the at least one cellular process operably linked to a light-inducible control sequence and (b) an ultrasound device.

32. A kit for regulating at least one cellular process in one or more cells comprising (a) one or more light-sensitive proteins capable of regulating the at least one cellular process or one or more constructs encoding the one or more light-sensitive proteins and (b) an ultrasound device.

33. A method of using one or more cells as a sensor for ultrasound intensity, comprising (a) introducing into the one or more cells a construct comprising a regulatory transgene capable of regulating at least one cellular process operably linked to a light-inducible control sequence or contacting the one or more cells with one or more light-sensitive proteins capable of regulating at least one cellular process; (b) exposing the one or more cells to ultrasound to inducing the expression of the regulatory transgene or to regulate the function of the one or more proteins using sonoluminescence (SL) or sonochemiluminescence (SCL); and (c) measuring a change in the at least one cellular process.
